(19) United States
(12) Patent Application Publication
(10) Pub. No.: US 2024/0091279 A1
(43) Pub. Date: Mar. 21, 2024

(54) COMBINATION THERAPY FOR INFLAMMATORY DISEASES

(71) Applicants: University of lowa Research Foundation, Iowa City, IA (US); The United States Government, as Represented by the Department of Veterans Affairs, Washington, DC (US)

(72) Inventors: Ashutosh Mangalam, Iowa City, IA (US); Shailesh K. Shahi, Iowa City, IA (US); Sudeep Ghimire, Iowa City, IA (US); Samantha Jensen, New York, NY (US)

(21) Appl. No.: 18/464,152

(22) Filed: Sep. 8, 2023

Related U.S. Application Data
(60) Provisional application No. 63/375,149, filed on Sep. 9, 2022.

(51) Int. Cl. A61K 35/741; A61K 31/353; A61P 37/06

(52) U.S. Cl. CPC A61K 35/741; A61K 31/353; A61P 37/06; A61K 2035/115

(57) ABSTRACT

A method to prevent, inhibit or treat an inflammatory disease in a mammal, is provided that includes administering to a mammal in need thereof an effective amount of one or more isolated gut microbes and one or more phytoestrogens.

### CROSS-REFERENCE TO RELATED APPLICATIONS

This application claims the benefit of the filing date of U.S. application No. 63/375,149, filed on Sep. 9, 2022, the disclosure of which is incorporated by reference herein.

### STATEMENT OF GOVERNMENT RIGHTS

This invention was made with government support under grant 1R01AI137075 awarded by the National Institutes of Health and 1101CX002212 awarded by the Department of Veteran Affairs. The government has certain rights in the invention.

### BACKGROUND

Multiple sclerosis (MS) is an autoimmune neurodegenerative disease of the central nervous system affecting around 2.8 million people worldwide which is 30% higher compared to the prevalence in 2013 (Walton et al., 2020). The etiopathogenesis of MS is complex and multifactorial involving both genetic and environmental factors (Mangalam et al., 2007; Koch et al., 2013; Giordano et al., 2002). The interplay between several factors leads to chronic inflammation of the CNS where the myelin sheath is broken down to manifest neurological symptoms including impairment of motor functions, vision, balance and sensitivity, depression, fatigue, cognitive changes, and gastrointestinal disorders. Genetic factors make up only about 30% of MS risk as shown by multiple previous studies (Willer et al., 2003; Hansen et al., 2005; Kuusisto et al., 2008) which provides strong support that environmental factors play a crucial role in the etiopathogenesis of MS. While there are numerous contributing environmental factors, we and others have previously demonstrated gut microbiota as an important factor associated with MS (Chen et al., 2016; Colpitts et al., 2017; Jangi et al., 2015). One of the key modulators of gut microbiota is diet. Dietary habits are reported to shape gut microbiota composition, intestinal inflammation, and immune status in the host (Maslowski & Mackay, 2011). Thus, a shift of specific micro/macronutrients modify the gut bacterial composition and the metabolite produced by them in the gut which in turn can influence gut barrier integrity and immune functions, host-microbe interactions, and skew the gut environment towards proinflammatory or anti-inflammatory responses (Rinninella et al., 2019, Forgie et al., 2019).

Isoflavones represent a major class of phyto-estrogenic compounds that are found in legume-based diets and possess numerous anti-oxidative, anti-bacterial, anti-carcinogenic, anti-inflammatory, and cardioprotective effects (Rimbach et al., 2008; Hertog et al., 1993; Larrosa et al., 2009). Some gut microbes such as Adlercreutzia equolifaciens (Maruo et al., 2008), Eggerthella species (Kim et al., 2009; Yokoyama & Suzuki, 2008), Slackia isoflavoniconvertens (Schroder et al., 2013) and Lactococcus garvieae (Uchiyama et al., 2007) specialize in metabolizing these phyto-estrogenic compounds. The key compounds daidzein and genistein of the isoflavone diet are metabolized by the gut microbiota to produce biologically active S-equol to exert numerous health benefits (Bowey et al., 2003).

### SUMMARY

The disclosure provides for the use of a phytoestrogen such an isoflavone and one or more commensal (e.g., gut) bacteria, for example, Parabacteroides, e.g., P. distasonis or P. gordini, and/or Adlercreutzia, e.g., A. equolifaciens, and/or Bifidobacterium, e.g., B. adolescentis, B. longum, B. pseudocatenulatum, B. stellenboschense, and B. thetataomicron, that is/are optionally lyophilized. The phytoestrogen such as an isoflavone and/or the one or more commensal bacteria may be provided in a tablet or capsule, e.g., both in a single capsule.

Thus, phytoestrogen and gut microbes may be used to prevent, inhibit or treat one or more symptoms of many diseases and can be used to boost health (as a synbiotic). In one embodiment, the diseases include but are not limited to autoimmune diseases such as multiple sclerosis, obesity, cancer and the like.

In one embodiment, the disclosure provides for a method to prevent, inhibit or treat an inflammatory disease, or one or more symptoms of an inflammatory disease, in a mammal, comprising: administering to a mammal in need thereof an effective amount of one or more isolated gut microbes and one or more phytoestrogens. In one embodiment, the mammal is a human. In one embodiment, the mammal has an autoimmune disease. In one embodiment, the autoimmune disease is multiple sclerosis. In one embodiment, at least one of the gut microbes comprises Parabacteroides. In one embodiment, one of the gut microbes comprises P. distasonis. In one embodiment, at least one of the gut microbes comprises Adlercreutzia species. In one embodiment, one of the gut microbes comprises A. equolifaciens. In one embodiment, one of the gut microbes comprises A. mucosicola. In one embodiment, one of the gut microbes comprises Bifidobacterium species. In one embodiment, one of the gut microbes comprises B. adolescentis. In one embodiment, one of the gut microbes comprises B. longum. In one embodiment, one of the gut microbes comprises B. thetataomicron. In one embodiment, one of the gut microbes comprises B. pseudocatemulatum. In one embodiment, one of the gut microbes comprises B. stellenboschense. In one embodiment, multiple gut microbes comprising two or more of P. distasonis, P. gordini. A. equolifaciens, A. mucosicola, B. adolescentis, B. longum, B. pseudocatenulatum, B. stellenboschense, and B. thetataomicron may be employed. In one embodiment, the one or more phytoestrogens comprise coumestan, coumestrol or 4-methoxycourmestrol. In one embodiment, one or more phytoestrogens comprise a stilbenoid such as resveratrol. In one embodiment, the one or more phytoestrogens comprise a prenylflavonoid, xanthohumol, isoxanthohumol, naringenin, 6-prenylnaringenin (6PN), 8-prenylnaringenin (8PN), 6,8-prenylnaringenin (6,8PN) or 8-geranylnaringenin (8GN). Gut bacteria specially Eubacterium limosum can convert isoxanthohumol into 8-prenylnaringenin (8PN) and demethylate some isoflavonoids (Possemiers et al 2006 PMID 16772450). In one embodiment, the one or more phytoestrogens comprise a lignan. Dietary lignans include but are not limited to secoisolariciresinol and matairesinol, which can be metabolized by gut bacteria to enterodiol and enterolactone. In one embodiment, the one or more phytoestrogens comprise a flavonol (e.g., kaempferc, icalin or quercertin). In one embodiment, the one or more phytoestrogens comprise an isoflavanoid, e.g., biochanin A, formononetin, glycitin, dihydrodaidzein, dihydrogenistein, O-desmethlangolensin (O-DMA), coumestrol or 4-methoxycourmestrol. In one embodiment, the one or more phytoestrogens comprise isoflavanol, genistein, quercetin, daidzin, daidzein or S-equol. In one embodiment, the one or more gut microbes and the one or more phytoestrogens are administered concurrently. In one embodiment, the one or more gut microbes are administered before the one or more phytoestrogens are administered. In one embodiment, the one or more gut microbes are administered after the one or more phytoestrogens are administered. In one embodiment, the one or more gut microbes are orally administered. In one embodiment, the one or more phytoestrogens are orally administered. In one embodiment, the mammal is administered a composition comprising the one or more gut microbes and the one or more phytoestrogens.

Further provided is a method to decrease disease severity in a mammal, comprising: administering to a mammal in need thereof an effective amount of one or more isolated gut microbes and one or more phytoestrogens. In one embodiment, the disease is an inflammatory disease. In one embodiment, the mammal is obese. In one embodiment, the mammal has cancer. In one embodiment, the mammal has an autoimmune disease. In one embodiment, the mammal is a human. In one embodiment, at least one of the gut microbes comprises Parabacteroides or Adlercreutzia. In one embodiment, the microbe(s) comprise P. acidifaciens, P. bouchesdurhonensis, P. chartae, P. chinchillae, P. chongii, P. distasonis, P. faecis, P. goldsleini, P. gordinii, P. johnsonii, P. massiliensis, P. merdae, P. pacaensis, P. provencensis or P. timonensis. In one embodiment, the one or more phytoestrogens comprise isoflavanol, genistein, quercetin, daidzein or S-equol, or any combination thereof. In one embodiment, the one or more gut microbes and the one or more phytoestrogens are administered concurrently. In one embodiment, the one or more gut microbes and/or wherein the one or more phytoestrogens are orally administered.

In one embodiment, a composition is provided comprising an amount of one or more isolated gut microbes and an amount of one or more phytoestrogens. In one embodiment, the composition is formulated for oral delivery. In one embodiment, The composition is formulated for sustained delivery. In one embodiment, at least one of the gut microbes comprises a bacterium. In one embodiment, at least one of the gut microbes comprise Parabacteroides. In one embodiment, one of the gut microbes comprise P. distasonis. In one embodiment, one of the gut microbes comprise P. gordini. In one embodiment, at least one of the gut microbes comprises Adlercreutzia. In one embodiment, one of the gut microbes comprises A. equolifaciens. In one embodiment, one of the gut microbes comprise A. mucosicola. In one embodiment, one of the gut microbes comprises B. adolescentis. In one embodiment, one of the gut microbes comprises B. longum. In one embodiment, one of the gut microbes comprises B. thetataomicron. In one embodiment, one of the gut microbes comprises B. pseudocatenulatum. In one embodiment, one of the gut microbes comprises B. stellenboschense. In one embodiment, the one or more phytoestrogens comprise isoflavanol, genistein, quercetin, daidzein or S-equol. In one embodiment, the one or more phytoestrogens comprise isoflavone, isoflavanol, isoflavan, isoflavanone, pterocarpan, flav-3-ene,3-arylcoumarin, moumaonchromone, counmaronchromene, formononetin, coumestrol, biochanin-A, pisatin, medicarpin, daidzein, daidzin, ononin, 4′-O-methylgeneistein, 4′-O-methyldaidzein, daidzein-7-O-phosphate or 3′-hydoxydaidzein. In one embodiment, the composition comprises P. distasonis, A. equolifaciens and an isoflavanoid.

In one embodiment, a package is provided comprising a first composition comprising an amount of one or more isolated gut microbes and a second composition comprising an amount of one or more phytoestrogens, wherein one of the microbes comprises Parabacteroides or Adlercreutzia. In one embodiment, the first composition, the second composition, or both, are formulated for oral delivery. In one embodiment, the first composition, the second composition, or both, are formulated for sustained delivery. In one embodiment, the gut microbes comprise Parabacteroides and Adlercreutzia. In one embodiment, the gut microbes comprise P. distasonis. In one embodiment, the gut microbes comprise A. equolifaciens. In one embodiment, one of the gut microbes comprises B. adolescentis. In one embodiment, one of the gut microbes comprises B. longum. In one embodiment, one of the gut microbes comprises B. thetataomicron. In one embodiment, one of the gut microbes comprises B. pseudocatenulatum. In one embodiment, one of the gut microbes comprises B. stellenboschense. In one embodiment, the one or more phytoestrogens comprise isoflavanol, genstein, quercetin, daidzein or S-equol.

As also described herein, the Akkermansia to Bifidobacterium ratio determines the pathobiont to symbiont feature of Akkermansia. A healthy phytoestrogen diet keeps a healthy state by enriching for beneficial bacteria such as Bifidobacterium adolescentis and the presence of these bacteria prevents or inhibits other commensals such as Akkermansia from becoming pathogenic. Thus, the disclosure provides a method comprising determining the ratio of Akkermansia to Bifidobacterium in a fecal sample from a mammal; and comparing that ratio to the ratio in a corresponding mammal that is on a healthy diet or to the ratio in a disease-free mammal, thereby determining the gut health of the mammal providing the fecal sample.

### BRIEF DESCRIPTION OF THE FIGURES

FIGS. 1A-1J. Diet change alters community composition and richness A) Experimental schematic outline for diet change and fecal sample collection. Alpha diversity measures B) Shannon diversity C) Faith's Phylogenetic diversity pre-diet change on day 0 from isoflavone rich (ISO) to phyto-free (PF) or vice versa. D) Shannon diversity and E) Faith's phylogenetic diversity changes after the diet was switched from PF to ISO or vice versa. Beta diversity at F) baseline (D0) and G) day 28 after diet change measured using weighted unifrac distance metrics. F) Beta diversity between D0 and day 28 (D28) for diet change from h) ISO to PF and I) PF to ISO. adonis2 test was performed to for statistical differentiation between the groups for beta diversity analysis. J) A network created using Jaccard dissimilarity matrix between the samples over time. The colors represent the different phases of microbial community change over time and the shape represents the change in diet.

FIGS. 2A-2B. Differentially abundant microbial taxa after the diet switch from either phyto-free (PF) to isoflavones (ISO) or vice-versa. Taxonomic differentiation of the fecal microbial taxa at A) baseline (D0) between the mice kept on ISO and PF diet. B) day 28 (D28) after diet switch from ISO to PF (ISO_PF) or PF to ISO (PF_ISO). Differential analysis was performed using welch test from microbiomeMarker package in R with p-value_cutoff of 0.01 and p_adjust using “BH”.

FIGS. 3A-3H. Scatterplots displaying correlation of normalized abundance of significantly altering taxa overtime after the diet change from isoflavone to phyto-free (ISO_PF) and phyto-free to isoflavones (PF_ISO). A positive or negative correlation is expressed by the values of correlation coefficient “R” calculated using “pearson” method with corresponding p values for both dietary changes.

FIGS. 4A-4F. Relative abundances of significantly altering active carbohydrate enzyme families identified using dbCAN2 and CaZy database in the metagenomes of mice before diet switch at day 0 and after diet switch at day 28. Diet change from isoflavone to phyto-free is represented as ISO_PF and phyto-free to isoflavones as PF_ISO. GTs represent glycosyltransferases, PLs represent polysaccharide lyases and SLHs represent S-Layer homology domain active carbohydrate enzymatic families.

FIGS. 5A-5D. Functional alteration of the gut microbiota A) at baseline (D0) between mice kept on either isoflavone (ISO) or phytofree (PF) and B) at day 28 (D28) after diet switch from ISO to PF (ISO_PF) and PF to ISO (PF_ISO). The differentially abundant pathways were identified using the welch test with pvalue_cutoff of 0.1, p_adjust=“BH” and nperm=1000 between the two groups. For the change in functional profile over time, D0 and D28 were compared using the welch test with pvalue_cutoff of 0.2, p_adjust=“BH” and nperm=1000 between the groups of C) diet change from ISO to PF and d) diet change from PF to ISO. The functional profile was obtained using human3 from the MetaCyc database.

FIGS. 6A-6H. Anti-inflammatory effect of LPS isolated from feces of mice kept on isoflavone diet. LPS was extracted from the feces of mice kept on phyto-free (PF) or isoflavone (ISO) diet for four weeks and was used to treat bone-marrow-derived macrophages. A) Fecal endotoxin levels were determined using LAL endotoxin assay. The concentration of B) TNF-□ C) IL-6 D) IL-12/23, E) IL-1□, F) CXCL1, and G) IL-10 was determined using ELISA. H) Ratio of IL-10 to IL-12/23. Mann-Whitney test was performed for (A) and (H) while Brown-Forsythe and Welch ANOVA tests followed by Dunnett T3 for multiple comparisons were performed for b, c, d, e, f and g to determine the statistical significance.

FIGS. 7A-7C. EAE severity in mice after diet switch from phyto-free (PF) to isoflavone (ISO) or ISO to PF diet. Mice were kept for six weeks on PF/ISO diet until day-28 where diet was switched for four weeks. After the diet switch, EAE was induced using MOG₃₅₋₅₅. A) Experimental outline of dietary regime and EAE induction in mice and clinical monitoring of EAE scores. B) Average clinical EAE scores of mice in which diet was changed from PF to ISO (PF_ISO, n=8) and ISO to PF (ISO_PF, n=10) for 41 days post EAE induction. C) Kaplan-Meier survival analysis of mice after EAE induction for 41 days.

FIG. 8. Concentration of S-equol in mice urine before and after the dietswitch (on day 28). PF_ISO and ISO_PF represent dietswitch from PF to ISO and ISO_PF respectively. Kruskal Wallis test followed by Dunn's multiple comparison was performed to determine the statistical significance.

FIGS. 9A, 9B and 9C-1-9C-4. Taxonomic investigation of the fecal microbiota over time after diet switch from phytofree (PF) to isoflavone (ISO): (PF_ISO) or ISO to PF (ISO_PF) A) Bar plot representing bacterial phylum with at least >1% relative abundance. B) Change in the Bacteroidetes:Firmicutes ratio (B:F ratio) overtime after the diet switch from either PF_ISO or ISO_PF. C) Bar plot representing species level classification with at least >5% relative abundance over time.

FIGS. 10A-10D. PCoA plots depicting differences in the diversity of functional pathways using “euclidean” as distance method from phyloseq package in R A) before diet change at day 0 (D0) between mice kept on isoflavone and phyto-free diet B) after diet change in mice from isoflavones to phyto-free (ISO_PF) and phyto-free to isoflavones (PF_ISO) at day 28 (D28) C) between D0 and D28 in mice where the diet was changed from phyto-free to isoflavones (PF_ISO) and D) between D0 and D28 in mice where the diet was changed from isoflavones to phyto-free (ISO_PF).

FIG. 11A-11C. Ratio of A. muciniphila (AM) to Bifidobacterium adolescentis (BA), B. longum (BL) and Bacteroides thetaiotamicron (BT) was calculated longitudinally starting at baseline (D0) till the duration of studies (28 days) after the diet switch from ISO to PF (ISO_PF) or PF to ISO (PF_ISO). The diet switch from ISO to PF (blue) resulted in an increased ratio of AM to BA, BL, and BT. Multiple t-tests were performed at each time. point to calculate p-value.

FIG. 12A-12C. Among the major differentially abundant bacteria that were lost when diet was switched from Iso-D to PF-D in the diet-switch study, only ratio of AM to B adolescentis (BA) was different between MS and HC. Fecal samples from MS (N=45) and HC (n=50) were analyzed by shotgun metagenomic sequencing using a method described previously.

### DETAILED DESCRIPTION

Lack of phytoestrogen metabolizing gut bacteria might lead to pathologies/diseases. Phytoestrogen and gut bacteria with ability to metabolize phytoestrogen allow for a beneficial effect. Thus, consumption of dietary phytoestrogen and phytoestrogen metabolizing bacteria provides for health benefits.

MS is a chronic, inflammatory demyelinating disease of the CNS resulting from an immune-mediated attack on myelinated axons (Hoftberger & Lassmann, 20107). MS pathogenesis results from multifactorial etiology involving both genetic and environmental factors (Goodin, 2009; Waubant et al., 2009). However, studies of monozygotic twins have shown that genetic factors contribute only ˜30% of the disease risk (Ebers et al., 1986), with the rest linked to environmental factors. Several environmental factors have been implicated in MS, including vitamin D deficiency, smoking, and exposure to certain infections, such as the Epstein-Barr virus (Waubant et al., 2019). In recent years, the gut microbiota has emerged as an important environmental factor linked with the pathobiology of MS (Chen et al., 2016; Cekanaviciute et al., 2016; Cosorich et al., 2017; Jangi et al., 2016; Miyake et al., 2015; Yadav et al, 2017; Cantoni et al., 2022).

Microorganisms inhabiting the gastrointestinal tract, collectively known as the “gut microbiota,” have evolved with the host over time, forming an intimate and mutually advantageous relationship. The host provides space and nutrients to these microorganisms while the gut microbiota helps with a number of host physiological processes such as the efficient digestion of food to extract energy, maintenance of gut barrier integrity, regulation of host immune responses, protection from pathogens, etc. (den Besten et al., 2013; Gensollen et al., 2016; Ghimire et al., 2020; Natividad & Verdu, 2013). During homeostasis, there is a symbiotic relationship between host factors and the microbiota (eubiosis) which helps in maintaining a healthy state. However, alterations in gut microbiota composition and function can perturb this homeostasis, known as gut dysbiosis, which has been reported in multiple diseases, including MS.

An isoflavone diet was shown to reduce the severity of MS in the animal model, experimental autoimmune encephalomyelitis (EAE). The studies imply that the gut microbiota composition of mice kept on phytoestrogen-free (“phyto-free”) diet are more similar to the gut microbiota composition of PwMS, specifically regarding bacteria capable of metabolizing isoflavones. As isoflavone metabolism is identified as one of the key microbial characteristics in healthy microbiota, it is important to understand whether diet change to isoflavones can modify the existing microbiota of PwMS to impart health benefits. To test the potential of isoflavone-containing diet for translation into the clinic, the PwMS microbiota was modeled by rearing mice on phytoestrogen free diet where mice lacked prevalent phytoestrogen metabolizing bacteria before switching them to a diet with isoflavone to evaluate its ability to suppress inflammation and disease.

The effect of change in the diet from phyto-free to isoflavone or vice-versa on gut microbiota composition, modulation of the immune response, and EAE disease severity was thus examined. It was observed that changing the diet from phyto-free to isoflavone resulted in modulation of gut microbiota taxonomic composition and functions. Specifically, it was observed that mice switched to an isoflavone diet showed overall improved gut health in the context of microbiome composition and stability compared to mice whose diet was changed to phyto-free. Functional investigation revealed differences in lipopolysaccharide biosynthesis after the diet change with LPS extracted from feces of isoflavone-fed mice inducing enhanced anti-inflammatory cytokine production from bone marrow-derived macrophages. Finally, it was observed that mice whose diet was switched from phyto-free to isoflavone diet showed reduced disease severity and mortality even though statistically significant levels were not reached at the time tested. Collectively, the data show an alternative pathway (alteration of LPS biosynthesis) through which a diet rich in isoflavones can induce an anti-inflammatory immune response and modulate CNS autoimmunity.

### Links Between the Gut Microbiome and MS

Multiple studies have shown that people with MS (PwMS) have microbial dysbiosis (Chen et al., 2016; 2016; Cekanavicuite et al., 2017; Cosorich et al., 2017; Jangi et al., 2016; Jangi et al., 2016; Miyake et al., 2015; Yadav et al., 2017; Cantoni et al., 2022). Specifically, PwMS have distinct gut microbiome communities compared to healthy individuals, characterized by an increased abundance of Pseudomonas, Mycoplasma, Haemophilus, Blautia, and Dorea and a decreased abundance of Parabacteroides, Prevotella, Adlercreutzia, Bacteroides, Faecalibacterium, Anaerostipes, Lachnospiracea, Butyricimonas and Lactobacillus (Chen et al., 2016; 2016; Cekanavicuite et al., 2017; Cosorich et al., 2017; Jangi et al., 2016; Jangi et al., 2016; Miyake et al., 2015; Yadav et al., 2017; Cantoni et al., 2022). A recent study from Denmark observed enrichment of 31 different bacterial species, such as Blautia, Ruminococcus, Bilophila, Sellimonas, etc. in PwMS, and these bacteria were linked to an increase in levels of inflammatory cytokines during disease activity (Thirion et al., 2023). Moreover, we and others have shown that gut resident fungi (mycobiome) are also modulated in PwMS (Yadav et al., 2022; Shah et al., 2021) characterized by a reduced abundance of Saccharomyces (Yadav et al., 2022), a fungus with probiotic-like properties. Thus, results from multiple microbiome studies strengthen the idea that PwMS have an altered microbiome compared to HC with depletion/reduced abundance of beneficial gut bacteria/fungi.

Although cross-sectional studies have helped establish a link between gut microbiota and MS, the question remains whether gut microbiota contributes to disease or disease causes dysbiosis or both. A recent study from Denmark showed a direct correlation between species richness and the number of disease relapses (Thirion et al., 2023). The clinically non-active (non-relapsing) patients showed enrichment of Faecalibacterium prausnitzii, Gordonibacter urolithinfaciens, Anaerostipes hadrus, Gemmiger formicilis, and Roseburia inulinivorans compared to clinically active patients (who had at least one relapse in follow up period). The clinically active group showed significant enrichment of Methanobrevibacter smithii and Victivallis vadensis. Interestingly, bacterial species more abundant in clinically active treatment-naïve cases were positively associated with circulating levels of proinflammatory cytokines IL-22, IL-17A, IFN-b, IL-33, and TNF-a. Thus, this study provides data that gut microbiota may contribute to MS disease severity by inducing a pro-inflammatory environment.

As microbiome studies suggest an association between gut microbiota and MS, there has been a strong emphasis on understanding the factors affecting the gut microbiota and the mechanism(s) through which gut microbes contribute to MS disease. Therefore, diet may regulate gut microbiota and affect the pathobiology of MS, using phytoestrogen metabolizing gut bacteria as an example.

### Environmental Factors, Gut Microbiota, and MS

Several external factors, such as geographical location, sun exposure, hygiene, and dietary habits, can influence the composition and function of the gut microbiota. Among these factors, diet has emerged as one of the most decisive non-host factors influencing gut microbiota composition (Rothschild et al., 2018; Qin et al., 2022). The human diet and gut microbiota have coevolved over millions of years, with changes in diet and lifestyle driving the evolution of the human gut microbiome. The diets of early humans were primarily composed of plant-based foods, such as fruits, vegetables, nuts, and seeds, and the occasional consumption of animal protein. A study comparing the microbiome of Hadza hunter-gatherers showed seasonal changes in the microbiome, especially wet season (increased plant food), characterized by the presence of bacteria linked with plant carbohydrate utilization (Smits et al., 2017). In contrast, mucin/animal carbohydrate-utilizing bacteria dominated the dry-season (increased hunting) microbiome. With the advent of agriculture and the cultivation of grains, legumes, and other crops, humans began consuming more carbohydrates (Smits et al., 2017; Brushett et al., 2020; Peddada, 2017; Swain & Ewald, 2018; Valle Gottlieb et al., 2018). This change in diet favored the growth of bacteria that could break down these new sources of carbohydrates. However, the industrialization of food production has led to the widespread consumption of processed and high-fat/high-sugar foods and the use of antibiotics (Swain Ewald & Ewald; 2018; Valle Gottlieb et al., 2018). This has contributed to decreased bacterial diversity and is associated with increased prevalence of diseases such as obesity, diabetes, inflammatory bowel disease, and MS. Interestingly, the microbiome of individuals from the USA showed a difference in mucin/animal carbohydrate utilization (Smits et al., 2017) and was similar to the dry season (meat-based diet) but significantly different from the wet-season microbiome of Hadza people, highlighting a loss of plant carbohydrate utilizing gut bacteria in industrialized nations. These evolutionary changes in the microbiome suggest that a diet promoting gut microbiota that utilizes plant carbohydrates might benefit the host, whereas a western diet promoting mucin degrader/animal carbohydrate-utilizing bacteria might predispose/propagate inflammatory diseases such as MS.

This hypothesis aligns with studies in PwMS and its animal model, experimental autoimmune encephalomyelitis (EAE), where a diet rich in fat or carbohydrates was linked with severe disease. Both obese individuals (Palmas et al., 2021, Shi et al., 2020) and PwMS (Tremlett et al., 2016; Chu et al., 2018; Camara-Lemarroy et al., 2018) showed gut dysbiosis with enrichment of the Desulfovibrionaceae family. Interestingly, mice on a high-fat diet (HFD) showed enrichment of bacteria from the Desulfovibrionaceae family, specifically Desulfovibrio piger and Bilophila wadsworthia. Additionally, HFD-induced obesity increased EAE disease severity in animal models of MS (Davanzo et al., 2023; Shahi et al., 2022; Ji et al., 2019). Besides high fats, Western diets are also rich in sugar, especially high fructose syrup. On a Fructose-rich diet (FRD), mice lost beneficial bacteria such as Prevotella, Muribaculum, and Bifidobacterium (Peterson et al., 2023). These FRD mice also showed an enrichment of Desulfovibrio, Colinsella, Olsenella, and Bacteroides as well as increased frequency of immune cells with pro-inflammatory characteristics (Peterson et al., 2023). These findings reinforce evidence from extensive population studies suggesting that the Western diet can lead to obesity and gut dysbiosis, which are, in turn, associated with severe disease in PwMS.

In contrast to humans, whose digestive systems generate roughly 17 gastrointestinal enzymes primarily for breaking down starch, the gut microbiota has thousands of supplementary enzymes (Human Microbiome Project, 2012; Sonnenburg & Backhed, 2016). This enables them to break down and ferment dietary polysaccharides into short-chain fatty acids (SCFAs) and other metabolites the host can absorb. Thus, a plant diet rich in microbiota-accessible carbohydrates (MAC) promote the growth of diverse beneficial gut microbiota linked with health. However, without plant-based carbohydrates, microbiota may utilize the glycoprotein-rich mucus layer as an alternative energy source. Only a select few gut bacteria, including Bifidobacterium and Akkermansia, have evolved the capacity to utilize mucin as a nutrient source (Derrien et al., 2008; Turroni et al., 2018a; Turroni et al., 2018b). As the mucosal layer and underlying epithelial cells play an important role in maintaining the barrier integrity (Allaire et al., 2018), plant carbohydrate-deprived microbiota would gradually begin to consume this protective barrier, resulting in inflammation and/or heightened vulnerability to pathogens (Breugelmans et al., 2022). In contrast, a plant-based diet rich in fiber/fermentable carbohydrate, phytoestrogen, tryptophan, and their metabolites, such as SCFA and equol, are linked with health. Thus, a well-balanced diet based on plant and unprocessed food can provide many health benefits by correcting gut dysbiosis. Multiple dietary regimens such as Mediterranean Diet, Paleolithic diet, modified paleo diet (Wahls diet), ketogenic diet, Swank diet, McDougall Diet, and fasting-mimicking diet are being explored as possible therapeutic strategies in PwMS (Stoiloudis et al., 2022: Wahls, 2022; Irish et al., 2017; Wahls et al., 2021). However, analysis of the gut microbiome in ongoing dietary intervention studies is needed to help establish whether these diets correct gut dysbiosis.

### The Gut Bacteria-Induced Phytoestrogen Metabolism and its Impact on the Regulation of Host Immunity

Phytoestrogens are plant-derived polyphenols with structural similarities to human estrogens and are comprised of several classes of chemical compounds, including isoflavones (soy), and lignans (flaxseed) (Branca & Lorenzetti, 2005; Cady et al., 2020). Humans don't have the capacity to metabolize phytoestrogen, but certain gut bacteria can metabolize them to produce metabolites such as S-equol (equol) from isoflavones, which shows significantly higher estrogenic activity than the original isoflavones (Morito et al., 2001). Estrogens have also been shown to possess immunomodulatory properties (Laffont et al., 2015; Offner & Polanczyk, 2006; Lang, 2004; McGill et al., 2020; Tiwari-Woodruff et al., 2007; Tiwari-Woodruff& Voskuhl, 2009) and ameliorate MS during pregnancy (Confavreux et al., 1998).

Several gut bacteria lacking in PwMS such as Prevotella, Parabacteroides, Adlercreutzia, and Bifidobacterium can metabolize dietary phytoestrogen. The significance of phytoestrogen and phytoestrogen metabolizing gut microbiota in disease protection was confirmed in EAE, as mice on a diet with isoflavones (a type of phytoestrogen) are protected from EAE (Jensen et al., 2021). Importantly, disease protection depended on the presence of gut microbiota as antibiotic depletion of microbiota abrogated the disease protective effect, and supplementation with phytoestrogen metabolizing bacteria restored disease protective function (Jensen et al., 2021). Additionally, mice on an isoflavone diet showed a lower inflammatory response as measured by antigen specific CD4 T cell proliferation, pro-inflammatory cytokine production, and increased levels of sera metabolites linked with an anti-inflammatory nature such as polyunsaturated fatty acids (Shrode et al., 2022). Additionally, the ability of exogenous equol to protect mice on a phytoestrogen-free diet confirmed the importance of gut bacteria-induced phytoestrogen metabolism in the EAE disease protection (Jensen et al., 2021).

The gut microbiome of mice on an isoflavone diet showed similarities with the microbiome of HC whereas the microbiome of mice on a phytoestrogen-free diet resembled the microbiome of PwMS characterized by a higher abundance of Akkermansia muciniphila (Jensen et al., 2021). Therefore, in a follow-up study (Ghimire et al., 2022), it was tested whether switching the diet from a phytoestrogen-free diet to an isoflavone diet would restore gut microbial homeostasis. Four- to six-week-old mice on standard chow were switched to an isoflavone-diet or phytoestrogen-free diet, and after six weeks on special diets, isoflavone-diet mice were switched to a phytoestrogen-free diet, and vice versa. The change from an isoflavone diet to a phytoestrogen-free diet reduced the overall richness and evenness of the microbial communities. However, a significant change in richness and evenness of the gut microbiota was not detected when the diet was changed from phytoestrogen-free to isoflavone-diet, suggesting that the absence of phytoestrogen strongly impacts the diversity of the gut microbiota.

Although the A. muciniphila levels did not change when switching diets, the change in diet from an isoflavone diet to a phytoestrogen-free diet significantly reduced the abundance of Bifidobacterium species, specifically B. adolescentis and B. longum (Ghimire et al., 2022). As Bifidobacterium are designated as beneficial “probiotic” species, (Sullivan & Nord 2005; Hidalgo-Cantabrana et al., 2017; Gibson, 1999; Chien et al., 2006) losing these species after the diet change to phytoestrogen-free indicates overall deterioration of gut microbiome homeostasis. Interestingly, the phytoestrogen metabolizing genes and equol levels were lost within a week after switching the diet from an isoflavone diet to a phytoestrogen-free diet (Ghimire et al., 2022). In contrast, when switching the diet from a phytoestrogen-free to an isoflavone diet, the phytoestrogen metabolizing genes and functions were restored after only 28 days of diet switch (Ghimire et al., 2022). Thus, switching to a phytoestrogen-free diet is associated with an inflammatory phenotype characterized by the loss of beneficial bacteria, especially Bifidobacterium species, without changes in the abundance of A. muciniphila. These data highlight the importance of diet in influencing the composition of gut microbiota. The next logical question is how these isoflavone-diet-induced changes in the composition of gut microbiota affect inflammation and disease.

### Dietary Change Alters Gut Microbial Lipopolysaccharide Biosynthesis and Modulates

Pro and Anti-Inflammatory Cytokine Production Analysis of bacterial functional pathways between the two diet groups showed a reduction in glycosyltransferase (GT) activity and an increase in polysaccharide lyase (PL) when the diet was changed from isoflavone to phytoestrogen-free diet (Ghimire et al., 2022). Both the GT and PL family of enzymes are involved in lipopolysaccharide (LPS) synthesis pathways, implying that isoflavone dietary conditions may contribute to the formation of structurally different LPS compared to a phytoestrogen-free diet. Interestingly, the LPS derived from the feces of mice kept on the phytoestrogen-free diet induced significantly higher levels of TNF-α, IL-1β, IL-6, IL-12/23, and CXCL1 compared to the LPS derived from feces of mice kept on the isoflavone-diet. On the other hand, IL-10 production was significantly higher in the mice kept on an isoflavone diet suggesting an immunoregulatory effect. Higher levels of pro-inflammatory cytokines TNF-α, IL-6, IL-1β, and IL-12/23 and lower levels of anti-inflammatory/immunoregulatory IL-10 indicate that the phytoestrogen-free diet may contribute to a pro-inflammatory effect in the gut. Finally, mice switched from an isoflavone diet to a phytoestrogen-free diet showed increased EAE severity compared to mice switched from a phytoestrogen-free to an isoflavone diet (Ghimire et al., 2022). Thus, an isoflavone-rich diet may protect from EAE by induction of an anti-inflammatory response through the enrichment of beneficial bacteria, equol production, and modulation of LPS biosynthesis pathways.

Interestingly, these data point towards a potential mechanism where the pro-inflammatory nature of a potential pathobiont such as A. muciniphila is determined by the presence or absence of other beneficial bacteria such as Bifidobacterium spp., modulation of LPS biosynthesis, and immunostimulatory capacity. This is an important finding regarding the symbiont versus pathobiont role of A. muciniphila and might explain the conflicting role of A. muciniphila reported in MS vs. obesity. While multiple studies, including the recent iMSMS study (iMSMS, 2022), showed enrichment of A. muciniphila in PwMS, A. muciniphila has also been shown to be a beneficial bacterium in obesity (Abuqwider et al., 2021; Yan et al., 2021). Interestingly, most Bifidobacterium spp. metabolize plant carbohydrates as a main food source, but some, including B. adolescentis, can also utilize mucin as a food source (Turroni et al., 2018b); Adamberg et al., 2018; Milani et al., 2016). In contrast, A. muciniphila is known for its ability to degrade and consume the mucin layer of the gut, but sometimes it can also metabolize other nutrients, including plant carbohydrates (Hagi & Belzer, 2021). Thus, a complex interaction among microbial community members foraging on similar food sources may influence the symbiont vs. pathobiont nature of specific bacterial strains. This might also explain the findings where the disease-protective effect of miR-30D was linked with the expansion of A. muciniphila (Liu et al., 2019). Based on our data, we hypothesize that the pathogenicity of A. muciniphila is determined by multiple factors, including diet and the absence of beneficial bacteria such as Bifidobacterium species, including B. adolescentis and B. longum.

### Exemplary Compositions

In one embodiment, the disclosure provides for a composition comprising one or more gut microbes and one or more phytoestrogens, or a composition comprising one or more gut microbes and a composition comprising one or more phytoestrogens, which is/are useful, in one embodiment, for the prevention, inhibition or treatment of inflammation or a disease or disorder that has inflammation.

In one embodiment, the composition is formulated for oral administration. In one embodiment, oral administration is achieved after compression into tablets or suspension of a lyophilized powder composition, e.g., comprising one or more gut microbes and one or more phytoestrogens, into a suitable oral vehicle, e.g., a capsule. In one embodiment, oral administration of the gut microbe(s) is achieved using live microbes. In one embodiment, the composition is formulated for subcutaneous, intravenous or intramuscular injection. In one embodiment, the use of the composition provides for therapeutic benefits in patients with, for example, an autoimmune disease or cancer, or in heathy individuals.

### Exemplary Uses for the Composition(s)

The composition(s) disclosed herein are useful to maintain health, to prevent, inhibit or treat one or more diseases or disorders, or to prevent, inhibit or treat one or more symptoms of one or more diseases or disorders.

In one embodiment, the composition(s) disclosed herein may be administered so as to prevent, inhibit or treat a disease in a mammal, or to prevent, inhibit or treat one or more symptoms of a disease in a mammal. Exemplary diseases include but are not limited to Alzheimer's disease, Parkinson disease, autism spectrum disorder, stroke, coronary heart disease, postmenopausal syndromes (hot flashes, bone resorptions), high cholesterol, or weight management (obesity).

For example, the composition(s) disclosed herein may be administered so as to prevent, inhibit or treat cancer in a mammal, or to prevent, inhibit or treat one or more symptoms of cancer in a mammal.

The term “cancer” is used to describe a proliferation of tumor cells (neoplasms) having the unique trait of loss of normal controls, resulting in unregulated growth, lack of differentiation, local tissue invasion, and/or metastasis. As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of dysplasia, or loss of differentiation and orientation of cells, and have the properties of invasion and metastasis. The term cancer also within context, includes drug resistant cancers, including multiple drug resistant cancers. Examples of neoplasms or neoplasias from which the target cell may be derived include, without limitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver (hepatocellular), lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others.

In one embodiment, the composition(s) disclosed herein may be administered so as to prevent, inhibit or treat an autoimmune disease in a mammal, or to prevent, inhibit or treat one or more symptoms of an autoimmune disease in a mammal. Exemplary autoimmune diseases include but are not limited to lupus erythematosus; Wiskott-Aldrich syndrome; autoimmune lymphoproliferative syndrome; myasthenia gravis; rheumatoid arthritis (RA); lupus nephritis; multiple sclerosis; optic neuritis; systemic lupus erythematosis; discoid lupus; subacute cutaneous lupus erythematosus; cutaneous lupus erythematosus including chilblain lupus erythematosus; chronic arthritis; Sjogren's syndrome; inflammatory chronic rhinosinusitis; colitis; celiac disease; inflammatory bowel disease; Barrett's esophagus; inflammatory gastritis; autoimmune nephritis; autoimmune vasculitis; autoimmune hepatitis; autoimmune carditis; autoimmune encephalitis; autoimmune diabetes; autoimmune diabetes nephritis; psoriasis; Graft-versus-host disease (GvHD); or autoimmune mediated hematological disease.

In one embodiment, the composition(s) disclosed herein may be administered so as to prevent, inhibit or treat a neurological disease in a mammal, or to prevent, inhibit or treat one or more symptoms of a neurological disease in a mammal.

### Exemplary Formulations

The disclosed composition(s) may include or may be formed from biodegradable polymeric molecules which may include, but are not limited to polylactic acid (PLA), polyglycolic acid (PGA), co-polymers of PLA and PGA (e.g., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

Nanoparticles, e.g., having one or more phytoestrogens, or microparticles, e.g., having one or more isolated bacteria or one or more phytoestrogens, may be prepared by methods known in the art. (See, e.g., Nagavarma et al., Asian J. of Pharma. And Clin. Res., Vol 5, Suppl 3, 2012, pages 16-23; Cismaru et al., Rev. Roum. Chim., 2010, 55(8), 433-442; and International Application Publication Nos. WO 2012/115806; and WO 2012/054425; the contents of which are incorporated herein by reference in their entireties). Suitable methods for preparing the nanoparticles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the nanoparticles may be prepared by forming a double emulsion (e.g., water-in-oil-in-water) and subsequently performing solvent-evaporation. The nanoparticles obtained by the disclosed methods may be subjected to further processing steps such as washing and lyophilization, as desired. Optionally, the nanoparticles may be combined with a preservative (e.g., trehalose).

Typically, the particles have a mean effective diameter of less than 1 micron, e.g., the nanoparticles have a mean effective diameter of between about 25 nm and about 500 nm, e.g., between about 50 nm and about 250 nm, about 100 nm to about 150 nm, about 150 nm to about 175 nm, about 150 nm to about 200 nm, about 400 nm to about 450 nm, or about 450 nm to 650 nm and the microparticles have a mean effective diameter of between about 25 μm and about 500 μm, e.g., between about 50 μm and about 250 μm, about 100 μm to about 150 μm, about 150 μm to about 175 μm, about 150 μm to about 200 μm, about 400 μm to about 450 nm, about 450 μm to 650 μm, about 550 μm to about 700 μm, about 600 μm to about 850 nm, or about 750 μm to 950 μm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

In one embodiment, a composition comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM, or any combination thereof. The disclosed polymer may include, but are not limited to, polyamidoamine (PAMAM) dendrimers. Polyamidoamine dendrimers suitable for preparing the presently disclosed nanoparticles may include 3rd-, 4th-, 5th-, or at least 6th-generation dendrimers.

In one embodiment, the delivery vehicle comprises a cationic lipid, e.g., N-[1-(2,3-dioleoyloxv)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N′-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; flies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, complexes are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, complexes are enteric coated or are embedded in micro-particle and enteric coated for delivery to gut.

Polymer based microparticles can be used to deliver compounds plus bacteria as disclosed herein and then enteric coated so that they are released in small intestine. In general, a suitable dose of one or more phytoestrogens may be in the range of from about 0.5 to about 50 mg/kg, e.g., from about 1 to about 25 mg/kg of body weight per day, such as 1 to about 20 mg per kilogram body weight of the recipient per day, for instance in the range of 0.5 to 20 mg/kg/day, e.g., in the range of 1 to 15 mg/kg/day. In general, a suitable dose of a bacterium or a combination of bacteria may be in the range from at least about 10⁴ to 10¹⁵ CFU, 10³ to 10¹⁴ CFU, 10⁶ to 10¹³ CFU, 10⁶ to 10¹² CFU, 10⁶ to 10¹³ CFU or 10⁷ to 10¹² CFU. Examples of polymers for the polymer based micro-particle include Poly(lactide-co-glycolide) (PLGA), Polylactic acid (PLA), Polycaprolactone (PCL), Gelatin, Dextran, Chitosan, Polyhydroxyalkanoates, Cellulose, Starch, Polyurethanes, Poly(vinyl chloride), Silanes, Methacrylates, and diaminosulfide polymer, poly(4,4′-trimethylenedipiperdyl sulfide) (PNSN). Once phytoestrogen and bacteria are loaded into microparticle, they can be enteric coated to protect them from degradation in acidic stomach environment and to be released in intestine.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

The biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

### Routes of Administration, Dosages and Dosage Forms

Administration of the composition(s) may be continuous or intermittent, depending, for example, upon the recipient's physiological condition, and other factors known to skilled practitioners. For example, initial treatment may be frequent (daily/alternate day) administration of phytoestrogen(s) and bacterium/bacteria which may be reduced after 4-8 weeks to once a week. The administration of the composition(s) may be essentially continuous over a preselected period of time or may be in a series of spaced doses. Both local administration and systemic administration are contemplated. Any route of administration may be employed, e.g., oral, intravenous, intranasal or intrabronchial, or local administration.

One or more suitable unit dosage forms comprising the composition(s), which may optionally be formulated for sustained release, can be administered by a variety of routes including parenteral, including by oral and rectal routes. The formulations may, where appropriate, be conveniently presented in discrete unit dosage forms and may be prepared by any of the methods well known to pharmacy. Such methods may include the step of bringing into association the active agent with carriers, solid matrices, semi-solid carriers, finely divided solid carriers or combinations thereof, and then, if necessary, introducing or shaping the product into the desired delivery system.

The amount of active ingredients in the composition(s) administered to achieve a particular outcome will vary depending on various factors including, but not limited to, the formulation, the condition, patient specific parameters, e.g., height, weight and age, and whether prevention or treatment, is to be achieved.

Compositions of the invention may conveniently be provided in the form of formulations suitable for administration. A suitable administration format may best be determined by a medical practitioner for each patient individually, according to standard procedures. Suitable pharmaceutically acceptable carriers (excipients) and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

Compositions of the present invention may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The compositions can be provided in a dosage form containing an amount effective in one or multiple doses. For example, the phytoestrogen may be administered in dosages of at least about 0.001 mg/kg to about 50 mg/kg, of at least about 0.001 mg/kg to about 25 mg/kg, at least about 0.01 mg/kg to about 20 mg/kg or at least about 0.5 mg/kg to about 15 mg/kg of body weight, although other dosages may provide beneficial results. For example, the gut bacteria may be administered in dosages of at least about 10⁴ to 10¹⁵ CFU, 10⁵ to 10¹⁴ CFU, 10⁶ to 10³ CFU, 10⁶ to 10¹² CFU, 10⁶ to 10¹³ CFU or 10⁷ to 10¹² CFU, although other dosages may provide beneficial results. The amount administered will vary depending on various factors including, but not limited to, the agent and/or carrier chosen for administration, the disease, the weight, the physical condition, the health, and/or the age of the mammal. Such factors can be readily determined by the clinician employing animal models or other test systems that are available in the art. As noted, the exact dose to be administered is determined by the attending clinician.

Pharmaceutical formulations can be prepared by procedures known in the art using well known and readily available ingredients. For example, the therapeutic agent can be formulated with one or more common excipients, diluents, or carriers, and formed into tablets, capsules, suspensions, powders, and the like. The compositions of the invention can also be formulated as elixirs or solutions appropriate for parenteral administration, for instance, by intramuscular, subcutaneous or intravenous routes.

The pharmaceutical formulations can also take the form of an aqueous or anhydrous solution, e.g., a lyophilized formulation, or dispersion, or alternatively the form of an emulsion or suspension.

In one embodiment, the compositions may be formulated for administration, and may be presented in unit dose form in tablets, capsules, ampules, pre-filled syringes, small volume infusion containers or in multi-dose containers. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

The formulations and compositions described herein may also contain other ingredients.

### Routes of Administration for Exemplary Compositions, and Dosages and Dosage Forms Therefor

Administration of compositions having one or more gut microbes, one or more phytoestrogens, or combinations thereof, and optionally another active, can be via any of suitable route of administration, particularly parenterally, for example, orally. Such administration may be as a single dose or multiple doses. For administration, the composition(s) may be formulated as a sterile solution in water or another suitable solvent or mixture of solvents, or in lyophilized form. The solution or lyophilized composition may contain other substances such as salts, sugars (particularly glucose or mannitol), to make the solution isotonic with blood, buffering agents such as acetic, citric, and/or phosphoric acids and their sodium salts, and preservatives.

A composition having one or more gut microbes, one or more phytoestrogens, or combinations thereof, or in combination with other active agents, can be formulated as pharmaceutical compositions and administered to a mammalian host, such as a human patient in a variety of forms adapted to the chosen route of administration, e.g., orally.

Thus, the compositions alone or in combination with another active agent, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the composition optionally in combination with another active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between, for example, about 2 to about 60% of the weight of a given unit dosage form. The amount of composition and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the phospholipid conjugate optionally in combination with another active compound may be incorporated into sustained-release preparations and devices.

The composition optionally in combination with another active compound or its salts can be prepared in water, optionally mixed with a nontoxic surfactant. Dispersions can also be prepared in glycerol, liquid polyethylene glycols, triacetin, and mixtures thereof and in oils.

The pharmaceutical dosage forms may include sterile aqueous solutions or dispersions or sterile powders comprising the active ingredient which are adapted for the extemporaneous preparation of sterile solutions or dispersions, optionally encapsulated in liposomes. In all cases, the ultimate dosage form may be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms during storage may optionally be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile solutions may be prepared by combining the one or more gut bacteria, one or more phytoestrogens, or combinations thereof, in the required amount, followed by filter sterilization. In the case of sterile powders for the, one method of preparation includes vacuum drying and the freeze drying techniques, which yield a powder of the active ingredient plus any additional desired ingredient.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and antimicrobial agents can be added to enhance the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers.

Useful dosages can be determined by comparing their in vitro activity, and in vivo activity in animal models. Methods for the extrapolation of effective dosages in mice, and other animals, to humans are known to the art.

Generally, the concentration of the microbe or phytoestrogen optionally in combination with another active compound in a composition, may be from about 0.1-25 wt-%, e.g., from about 0.5-10 wt-%. The concentration in a semi-solid or solid composition such as a gel or a powder will be about 0.1-5 wt-%, e.g., about 0.5-2.5 wt-%.

The amount of the microbe(s), phytoestrogen(s), or combinations thereof, optionally in combination with another active compound, or an active salt or derivative thereof, required for use in treatment may vary with the route of administration, the nature of the condition being treated and the age and condition of the patient and will be ultimately at the discretion of the attendant physician or clinician. In general, however, a suitable dose of one or more phytoestrogens may be in the range of from about 0.5 to about 50 mg/kg, e.g., from about 1 to about 25 mg/kg of body weight per day, such as 1 to about 20 mg per kilogram body weight of the recipient per day, for instance in the range of 0.5 to 20 mg/kg/day, e.g., in the range of 1 to 15 mg/kg/day. In general, a suitable dose of a bacterium or a combination of bacteria may be in the range from at least about 10⁴ to 10¹⁵ CFU, 10⁵ to 10¹⁴ CFU, 10⁶ to 10¹³ CFU, 10⁶ to 10¹² CFU, 10⁶ to 10¹¹ CFU or 10⁷ to 10¹² CFU.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The dose, and perhaps the dose frequency, will also vary according to the age, body weight, condition, and response of the individual patient. In general, the total daily dose range for microbe(s), phytoestrogen(s), or combinations thereof, for the conditions described herein, may be in single or divided doses. In one embodiment, a daily dose range for phytoestrogen may be about 20 mg to about 1000 mg, e.g., about 50-500 mg, in single or divided doses, e.g., 50 to 100 mg of orally administered phytoestrogen(s) In one embodiment, a daily dose range for bacteria may be about 10⁴ to 10¹⁵ CFU, e.g., about 10⁵ to 10¹⁴ CFU, in single or divided doses, e.g., 10⁶ to 10² CFU of orally administered bacteria. In managing the patient, the therapy may be initiated at a lower dose depending on the patient's global response.

The invention will be further described by the following non-limiting examples.

### Example I

The etiopathogenesis of multiple sclerosis (MS) is strongly affected by environmental factors such as diet and gut microbiota. Certain bacteria were depleted or enriched in patients with MS (pwMS), and it was found that a number of bacteria lacking in pwMS are responsible for metabolizing phytoestrogen, a chemical found in certain plant/diet such as soy and legume. In a mouse model of MS, it was observed that mice on a diet lacking phytoestrogen develop a severe form of disease, however those on a diet with isoflavone (a phytoestrogen found in soy) develop milder disease suggesting that isoflavone dampens inflammation thus suppressing the disease. In addition, an isoflavone diet loses it disease protective effect in the absence of gut bacteria (e.g., which were depleted using antibiotics) which suggests that both diet and bacteria are required for this protective effect. Additionally, mice placed on a diet with isoflavone cause an increase in two of the bacteria lacking in pwMS. When those bacteria are reintroduced, the protective effect of an isoflavone diet is restored, however, those bacteria have no effect when isoflavone/phytoestrogen is not present in diet. Equol (one of the potential metabolites bacteria produced after metabolizing isoflavone) can mimic some of the disease protective function.

An isoflavone diet was thus shown to reduce the severity of MS in the animal model, experimental autoimmune encephalomyelitis (EAE). Translation of this concept to the clinic would include providing the isoflavone-rich diet to people with MS (PwMS) who lack phytoestrogen metabolizing bacteria. As gut microbiota of PwMS resembles the gut microbiota of mice raised under phyto-free diet in terms of lack of phytoestrogen metabolizing bacteria, the diet of mice raised under phyto-free diet was switched to isoflavone diet to investigate the effects on microbiota, inflammatory response, and EAE disease severity. Microbiota analysis showed that the change in diet from isoflavone to phyto-free reduced beneficial bacteria such as Bifidobacterium species. Such alteration of the gut microbiota composition was observed along with functional differences in lipopolysaccharide biosynthesis pathways. Moreover, lipopolysaccharide (LPS) extracted from feces of isoflavone-fed mice was able to induce anti-inflammatory cytokine production from bone marrow-derived macrophages compared to fecal-LPS from phyto-free group. Eventually, mice whose diet was switched from phyto-free to isoflavones trended towards reduced EAE severity and mortality. Overall, the data show an alternative mechanism specifically modulation of LPS biosynthesis besides S-equol production by gut bacteria through which diet switch to isoflavones imparts an anti-inflammatory response including reduced disease severity.

The studies imply that the gut microbiota composition of mice kept on phytoestrogen-free (“phyto-free”) diet are more similar to the gut microbiota composition of PwMS, specifically regarding bacteria capable of metabolizing isoflavones. As isoflavone metabolism is identified as one of the key microbial characteristics in healthy microbiota, it is important to understand whether diet change to isoflavones can modify the existing microbiota of PwMS to impart health benefits. To test the potential of isoflavone-containing diet for translation into the clinic, the PwMS microbiota was modeled by rearing mice on phytoestrogen free diet where mice lacked prevalent phytoestrogen metabolizing bacteria before switching them to a diet with isoflavone to evaluate its ability to suppress inflammation and disease.

### Example 11

Using experimental autoimmune encephalomyelitis (EAE): a mouse model of MS, it was shown that gut microbiota is required for isoflavone diet mediated disease suppression (Jensen et al., 2021). The same study demonstrated that isoflavone metabolizing bacteria specifically Adlercreutzia equolifaciens and Parabacteroides distasonis, and/or their metabolite S-equol are sufficient to ameliorate EAE. Moreover, people with MS (PwMS) have a reduced abundance of isoflavone metabolizing bacteria such as A. equolifaciens and P. distasonis in their fecal samples compared to healthy controls (Chen et al., 2016).

### Materials and Methods

### Mice Procurement and Dietary Regiment

C57BL/6J female mice (4 to 6 weeks old) were purchased from Jackson Laboratories (Bar Harbor, ME) and maintained at the University of Iowa mice facility. Pups at the age of 4-6 weeks were placed on either an isoflavone diet [genistein (0.24 g/kg) and daidzein (0.22 g/kg of diet)] (n=10) or a phyto-free diet (Envigo, Indianapolis, IN) (n=8) ad libitum for 6 weeks before the diet switch was made.

### Fecal Sample Collection, Sequencing

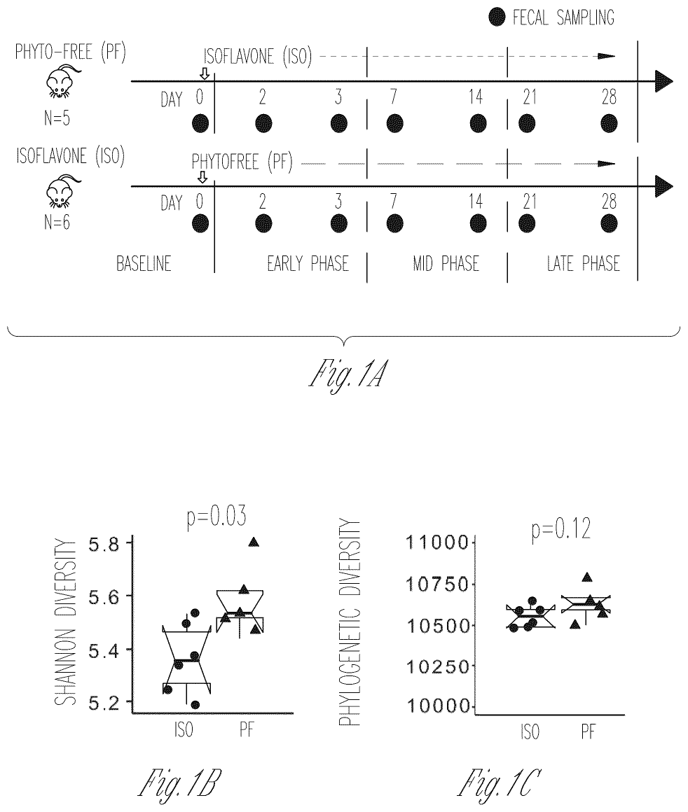

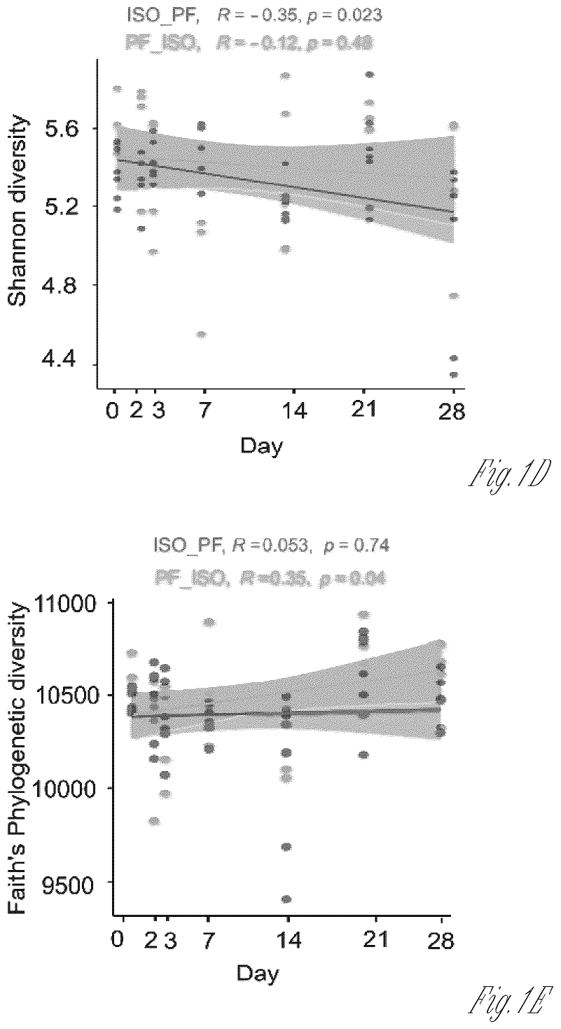

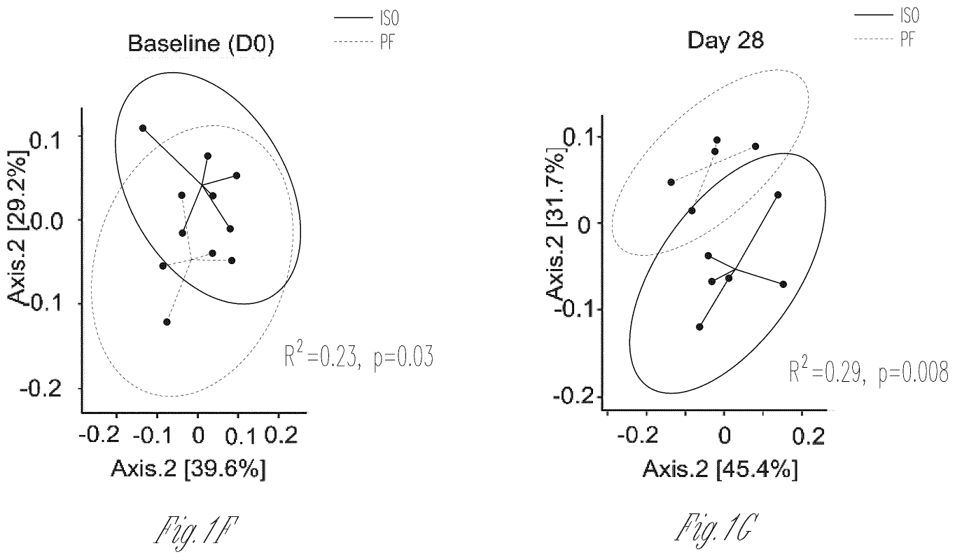

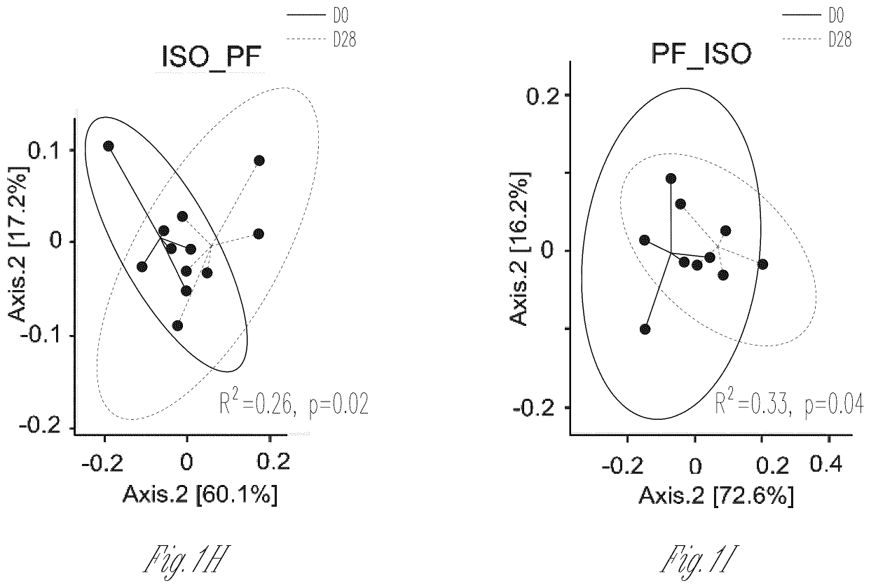

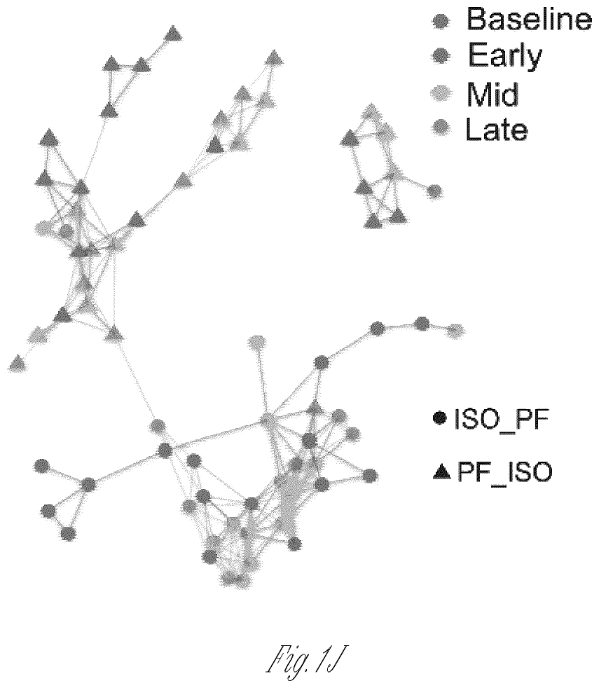

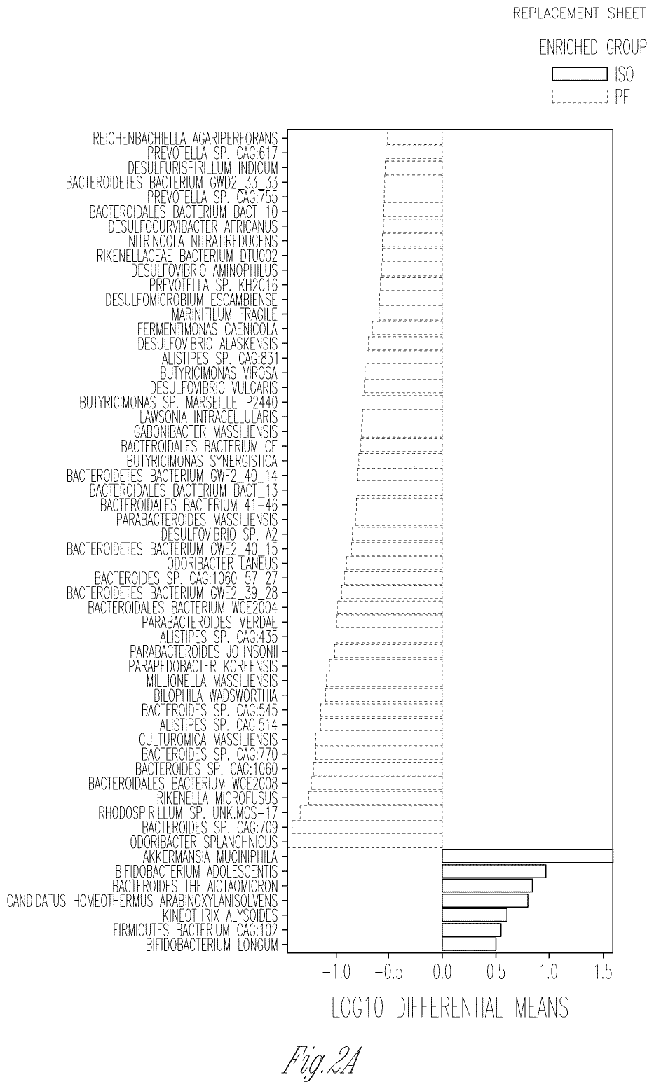

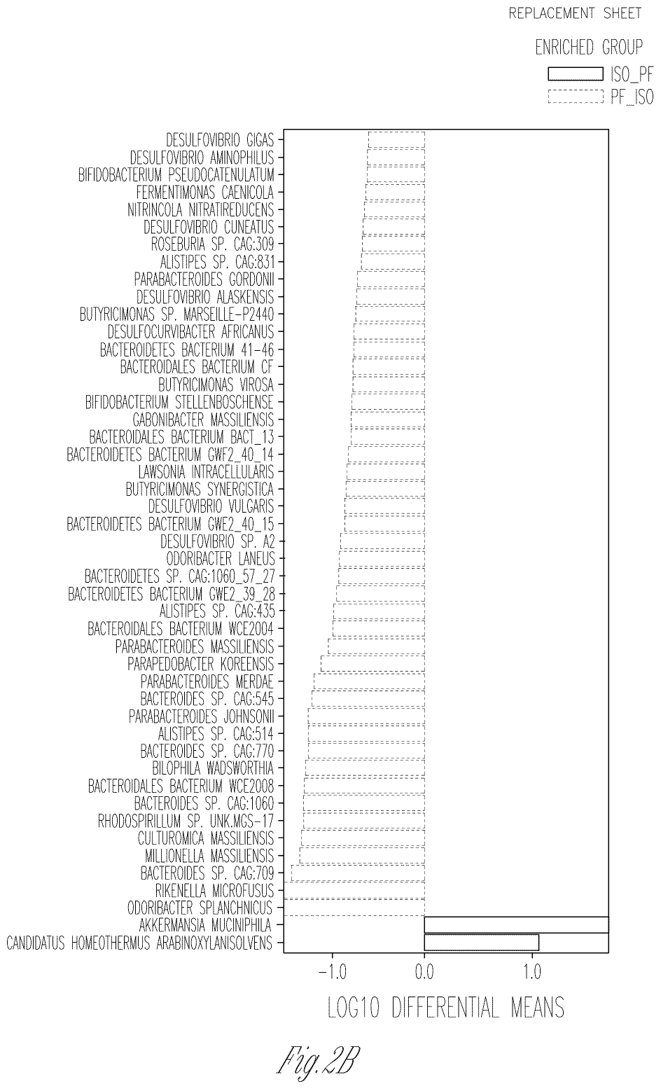

Fecal pellets from six individual mice were collected at different time points as shown in FIG. 1a. After the diet switch, mice were reared for 4 weeks before euthanasia. A total of 77 fecal pellet samples were collected on: D0 (baseline), and after diet switch on day 2, day 3, day 7, day 14, day 21 and day 28 and stored at −80° C. until further analysis. DNA was isolated using DNesay PowerLyzer PowerSoil Kit (Qiagen) following the manufacturer's instructions. DNA samples were quantified using Qubit (Qiagen) and sequenced using MiSeq Illumina platform in CosmosID Inc (Rockville, MD). The raw sequences were quality controlled using metawrap v=−0.3.2 read qc pipeline (Uritskiy et al., 2018). Briefly, adaptor sequences were trimmed, low-quality bases (Phred score <20) were removed, and host reads were removed by mapping reads to reference mouse genome (mm39) using default parameters. After quality control and host reads removal, the clean reads were mapped to proGenomes reference database (Mende et al., 2017) for taxonomic assignment of the sequences using Kaiju greedy mode (Menzel et al., 2016).

### Microbiota Analysis

The downstream analysis of the taxonomic table was performed in R v4.1.0 (R Core Team, 2021) using phyloseq, microbiome, and vegan packages after removing the reads assigned to either “unclassified”, “viruses” or “cannot be assigned to non-viral species”. One of the samples from the PF group was removed because of low number of sequences. So, the microbiota analysis includes 5 mice from PF and 6 mice from isoflavone group respectively for the downstream analysis. Shannon diversity and Faith's phylogenetic diversity were calculated as alpha diversity measures. The taxa were then filtered by removing those that are not observed at least 100 times in at least 20% of the samples and normalized to median sequencing depth. Weighted unifrac distances and Bray-Curtis dissimilarity metrics were used for beta diversity analysis. For time points, D0 (baseline), day 2, day 3, day 7, day 14, day 21 and day 28 were considered based on fecal collection. Differential microbiota analysis was performed using the Welch test from the microbiomemarker package (Yang, 2020). HUMANn3 (Beghini et al., 2021) was used with default parameters to profile functional pathway abundances in the clean reads. Pathways that were not observed at least 100 times in at least 10% of the samples were removed, normalized to median depth, and analyzed downstream in R.

### Gene Homology

Multiple microbial species are known to possess isoflavone metabolizing daidzein reductase (DR), dihydrodaidzein reductase (DHDR), and tetrahydrodaidzein reductase (TIHDR) genes. The nucleotide sequences of these genes from Adlercreutzia equolifaciens (DR: RFT81436.1, DHDR: RFT81438.1, THDR: RFT81439.1), Eggerthella sp. YY7918 (DR: BAK44713.1, DHDR: BAK44715.1, THDR: BAK44716.1), Lactococcus garvieae (DR: BAJ72750.1, DHDR: BAJ72748.1, THDR: BAJ72744.1), Senegalimassilia sp. KGMB04484 (DR: RXZ54824.1, DHDR: RXZ54822.1, THDR: RXZ54821.1), Slackia equolifaciens (DR: RNL39925.1, DHDR: RNL39927.1, THDR: RNL39927.1), Slackia sp. NATTS (DR: BAL46930.1, DHDR: BAL46929.1, THDR: BAL46928.1) and Slackia isoflavoniconvertens (DR: AFV15453.1, DHDR: AFV15451.1, THDR: AFV15450.1), the disclosures of which are incorporated by reference herein, were obtained from NCBI. The daidzein and genistein reductase (dgr) gene (KJ452760.1) from Slackia sp. AUH-JLC159 were obtained and combined with DR, DHDR, and THDR genes from the aforementioned bacterial species to create a custom database. The assembled fecal metagenomes from each mouse were searched for the presence of isoflavone metabolizing genes in a custom database using mucmer with default parameters (Marcais et al., 2018). Similarly, bacterial reference genomes of B. adolescentis (NZ_CP028341.1), B. longum (NZ_AKCA01000001.1), and B. stellenboschense (NZ_JGZP01000001.1) were obtained from NCBI and searched for the presence of isoflavone metabolizing genes.

Carbohydrate metabolizing active enzymes annotation and quantification Carbohydrate metabolizing active enzymes were predicted from the shotgun metagenomic sequences. After quality control and host reads removal, clean paired reads were assembled using Megahit using default parameters (Li et al., 2015). The obtained assembly of each sample was annotated using Prokka (Seemann, 2014) and .fna was used for identifying the active carbohydrate metabolizing enzymes in dbCAN2 with default parameters (Zhang et al., 2018). dbCAN2 aligns the sequences to CAZy database (Drula et al., 2022) to identify the enzymatic families. All enzymes identified by at least one of the HMMR, Hotpep, or DIAMOND algorithms within dbCAN2 were used for analysis. The relative abundance of the obtained modules was further analyzed using an unpaired t-test with Welch correction in GrapPad Prism 10.0.

Extraction of LPS from Fecal Lysate

Fecal samples were collected from mice (n=5) kept on phyto-free or isoflavone diet for 4 weeks. Lipopolysaccharide was isolated from the fecal samples following methods described elsewhere with slight modifications (Kim et al., 2016). Briefly, 100 mg of feces was mixed with 25 ml of PBS in a pyrogen-free tube and sonicated for 1 hour. The mixture was centrifuged at 400 g for 15 minutes and 15 ml of supernatant was collected. The supernatant was sterilized by filtration through a 0.22 μm non-positively charged filter and inactivated for 10 minutes at 70° C. The filtrate was then kept at −80° C. until further use. The fecal lysate was thawed and endotoxin levels were quantified in 10 μl of filtrates using ToxinSensor Chromogenic LAL Endotoxin Assay Kit (Cat. No. L00350, GenScript, NJ) following manufacturer's protocol.

Bone Marrow-Derived Macrophages Culture and Treatment with Fecal LPS

Bone marrow extraction and subsequent culture to yield macrophages were performed following methods previously described (Bailey et al., 2020). Briefly, mice were euthanized by exposure to carbon dioxide and bone marrow was extracted from femur and tibia bones aseptically. Clumps of marrow were gently disintegrated using a needle-less syringe and passed through a 70 μm cell strainer. The cell suspension was then centrifuged at 250 g for 5 minutes at room temperature, the supernatant was discarded, and the cells were suspended in DMEM F12 medium containing L-glutamine (2 mM), penicillin (100 units/ml), streptomycin (0.1 mg/ml), 5% FBS and recombinant MCSF (25 ng/ml) growth factor (DMEM F12 MCSF growth media). 200,000 cells/well were seeded into a non-tissue culture treated 12 well plate in 1 ml DMEM F12 MCSF growth media on D0. The cells were cultured for 7 days and fed with an addition of 500 μl of DMEM F12 MCSF growth media containing MCSF (50 ng/ml) on day 5 of culture. On day 7, the cells were washed with PBS to remove unadhered cells and 1 ml of media containing DMEM F12 containing 1-glutamine (2 mM), penicillin (100 units/ml), streptomycin (0.1 mg/ml) was added. Cells were then either left unstimulated (control) or were stimulated with 10 μl of LPS isolated from the fecal lysate of mice kept on phyto-free or isoflavone diet or 10 μl of LPS standard control solution (0.6 μg/μl) (Lipopolysaccharide Escherichia coli 055.B5, EMD Chemicals, CA) for 3 hours at 37° C. Subsequently, the cells were washed with PBS and the media was replaced with DMEM F12 containing 1-glutamine (2 mM), penicillin (100 units/ml), streptomycin (0.1 mg/ml) and incubated at 37° C. for 16 hours before collecting supernatants. The supernatants collected from all four groups; control, PF, ISO and LPS standard treated group were kept at −80° C. until further use. The levels of cytokines TNF-alpha, IL-6, IL-12/23 (Biolegend, CA), and IL-10 (BD Biosciences Pharmigen, CA) were quantified in the supernatants using ELISA as per manufacturer's instructions. GrapPad Prism 10.0 was used for visualization and statistical tests.

### S-Equol Quantification

S-equol was quantified from urine of the mice before and after 28 days of dietswitch. Urine samples were collected and sent to Metabolomics Core, Henry Ford Health System (Detroit, MI) for quantification using Liquid Chromatography Mass Spectrometry (LC-MS/MS) where S-equol was quantified following methods described previously with modifications (Raju et al., 2019). Briefly, Mobile phase A (2 mm Amonium acetate+0.2% acetic acid) and B (Acetonitrile+0.2% acetic acid) at a flow rate of 0.30 ml/minute used to separate the metabolites in gradient mode. Best separation of S-Equol was achieved using Waters Acquity Column, CSH 1.7 μm, 2.1 mm×150 mm kept at 65° C. using 5 minutes linear gradient. The column effluent was monitored by Negative Electrospray Ionization (ESI−) using multiple reaction monitoring (MRM) workflow. S-Equol peak area under the curve (AUC's) of the analyte was then used for quantification.

### EA Induction and Scoring

For EAE studies, EAE was induced and evaluated as described previously (Tyler et al., 2013). Briefly, mice were immunized subcutaneously on D0 on the left and right flank with 100 μg of MOG₃₅₋₅₅ emulsified in 200 μg of CFA followed by 80 ng of pertussis toxin (PTX) intraperitoneally on D0 and D2. Disease severity was scored as follows: 0, no clinical symptoms; 1, loss of tail tonicity; 2, hindlimb weakness: 3, hindlimb paralysis: 4, forelimb weakness; and 5, moribund or death.

All procedures were done according to the Institutional Animal Care and Use Committee guidelines at the University of Iowa.

### Results

### Diet Change to Isoflavones Enhances Microbial Diversity

To test if the change in diet can alter microbial evenness and diversity, the alpha diversity of the microbiome on baseline (D0) and on days 2, 3, 7, 14, 21, and 28 post-diet-switch was examined as shown in (FIG. 1a). On D0, fecal microbial communities of mice on PF diet had significantly higher Shannon diversity compared to mice on the ISO diet (p=0.03) (FIG. 1b). The PF diet also supported higher phylogenetically diverse microbes but was statistically non-significant compared to the ISO diet (p=0.12) (FIG. 1c). However, Shannon diversity of mice kept on the ISO diet was significantly reduced over time after the diet was switched to the PF indicating that diet change from ISO to PF reduced the overall richness and evenness of the microbial communities (FIG. 1d). Such significant change in the microbial community was not detected when the diet was changed from PF to ISO over time (p=0.48) suggesting that the ISO diet supported the growth of the diverse microbial species present in the PF diet at D0 over 28 days. Further, there was a significant rise in the phylogenetic diversity when the diet was switched from PF to ISO (p=0.04) implying that the diet change to ISO incorporated phylogenetically diverse microbes in the community over time which was not evident in the reverse from ISO to PF (p=0.74) (FIG. 1e). Furthermore, the fecal microbial communities of the mice kept on ISO and PF diets were significantly different from one another at baseline (FIG. 1f) and after day 28 (D28) of the diet switch (FIG. 1g). The change in diet from ISO to PF or PF to ISO significantly altered the community on D28 compared to D0 (FIG. 1h, 1i). There were no differences in the homogeneity of variances when the diet was switched from PF to ISO (p=0.71) or ISO to PF (p=0.68) indicating that samples were more even within the group. The diet-driven microbial community changes were evident for both alpha and beta diversity but the microbial communities on D28 did not radically change to be similar to either ISO or PF baseline (D0) community (FIG. 1j) indicating that dietary change can only prime the microbiota originally present. Thus, these data indicate that diet change to ISO enhances microbial diversity and evenness in the gut, a sign of overall better gut health.

### Diet Change to Phyto-Free Diet Reduces Beneficial Gut Bacteria

As diet alone can shape the microbiota, it was investigated if mice originally kept on PF diet and later switched to ISO diet can enrich for isoflavone metabolizing genes and the related bacteria. Isoflavone metabolism by gut microbiota is known to produce the metabolite S-equol that exerts numerous health benefits (Mayo et al., 2005). Thus, levels of S-equol in urine as well as the presence of isoflavone metabolizing genes in the assembled metagenomes at baseline through the end of the experiment were determined (FIG. 8, Table 1). Phytoestrogen metabolizing genes were completely absent in mice kept on PF diet on D0 whereas all mice kept on ISO diet had at least one gene homolog linked to phytoestrogen metabolism except for one mouse. This finding resonated with the S-equol levels where we observed no measurable quantity of S-equol in urine of mice kept on PF diet and significantly higher levels of S-equol in urine of mice kept on ISO diet before the diet switch (FIG. 8). This demonstrates the presence of phytoestrogen metabolizing bacteria in the metagenomes of mice kept on the ISO diet at the baseline. However, after the diet was changed from PF to ISO, the microbial community evolved to harbor phytoestrogen metabolizing gene after 28 days in two out of five mice and the S-equol levels were restored to similar levels of S-equol in mice kept on ISO diet pre diet change (FIG. 8). On other hand, the phytoestrogen metabolizing genes as well as S-equol levels were lost after the diet was switched to ISO to PF indicating that phytoestrogen metabolism capability and the associated function is lost after the diet change (Table 1).

Table 1: Identification of homologs of isoflavone metabolizing genes in metagenomes of feces of mice. Mice were kept initially on phyto-free or isoflavone diet for six weeks until day 0. Subsequently, on day 0, diet was switched between the groups to isoflavone from phyto-free (PF_ISO) or from isoflavone to phyto-free (ISO_PF). The assembled fecal metagenomes from each mouse were searched for the presence of isoflavone metabolizing genes in a custom database (see Materials and methods section) using rnucmer with default parameters (Marcais et al., 2018). Numbers inside parentheses represent the percentage identity of the individual homolog of isoflavone genes identified in the assembled fecal metagenomes of individual mice over time after the diet change.

| | Diet | Day after diet change | | | | | | |
|---|---|---|---|---|---|---|---|---|
| Mouse | change | 0 | 2 | 3 | 7 | 14 | 21 | 28 |
| BL01 | PF_ISO | — | — | — | — | — | — | — |
| BL02 | PF_ISO | — | — | — | — | — | — | — |
| BL03 | PF_ISO | — | — | — | — | — | — | — |
| BL04 | PF_ISO | — | — | — | — | — | — | DHDR |
| | | | | | | | | (97.63%), |
| | | | | | | | | THDR |
| | | | | | | | | (95.52%) |
| BL05 | PF_ISO | — | — | — | — | — | — | DHDR |
| | | | | | | | | (94.56%), |
| | | | | | | | | THDR |
| | | | | | | | | (91.94%) |
| BL09 | ISO_PF | DHDR | — | — | — | — | — | — |
| | | (97.23%), | | | | | | |
| | | THDR | | | | | | |
| | | (95.74%), | | | | | | |
| | | THDR | | | | | | |
| | | (95.76%) | | | | | | |
| BL10 | ISO_PF | — | DR | DHDR | — | — | — | — |
| | | | (96.1%) | (97.82%), | | | | |
| | | | | THDR | | | | |
| | | | | (94.67%), | | | | |
| | | | | THDR | | | | |
| | | | | (94.55%) | | | | |
| BL11 | ISO_PF | THDR | — | — | — | — | — | — |
| | | (92.94%) | | | | | | |
| BL12 | ISO_PF | DR | — | — | — | — | — | — |
| | | (91.65%), | | | | | | |
| | | DHDR | | | | | | |
| | | (97.38%), | | | | | | |
| | | DHDR | | | | | | |
| | | (91.45%), | | | | | | |
| | | THDR | | | | | | |
| | | (95.7%) | | | | | | |
| BL13 | ISO_PF | DR | — | — | — | — | — | — |
| | | (97.88%), | | | | | | |
| | | DR | | | | | | |
| | | (95.95%), | | | | | | |
| | | DHDR | | | | | | |
| | | (96.36%), | | | | | | |
| | | THDR | | | | | | |
| | | (93.71%) | | | | | | |
| BL16 | ISO_PF | DR | — | — | — | — | — | — |
| | | (99.49%), | | | | | | |
| | | DR | | | | | | |
| | | (96.28%), | | | | | | |
| | | DHDR | | | | | | |
| | | (97.06%), | | | | | | |
| | | DHDR | | | | | | |
| | | (88.24%), | | | | | | |
| | | THDR | | | | | | |
| | | (94.88%), | | | | | | |
| | | THDR | | | | | | |
| | | (92.70%) | | | | | | |

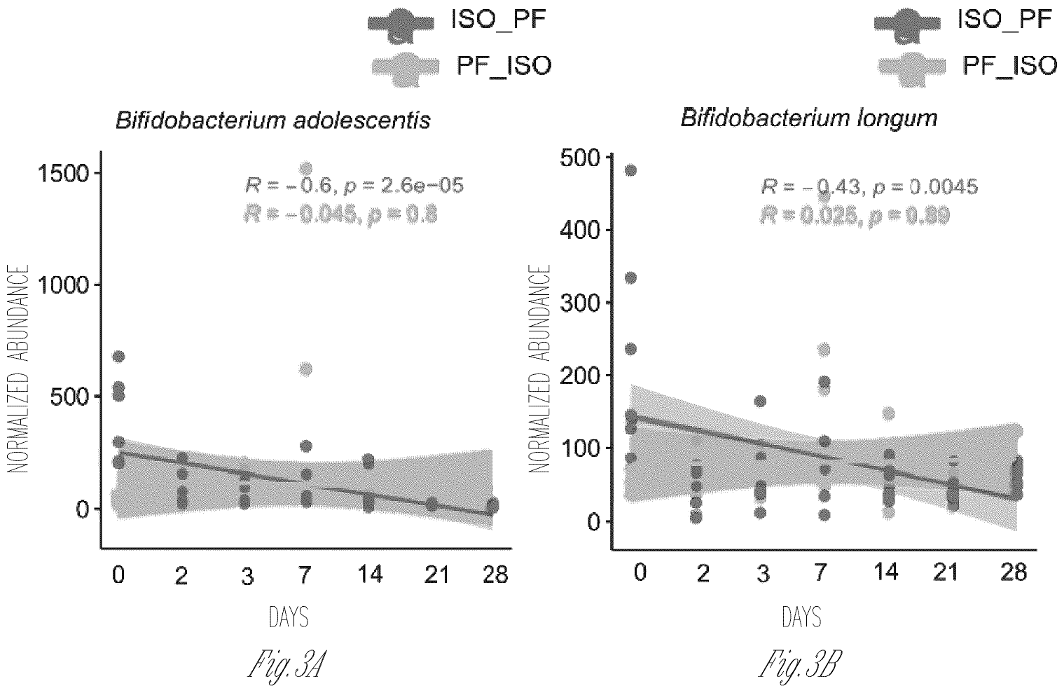

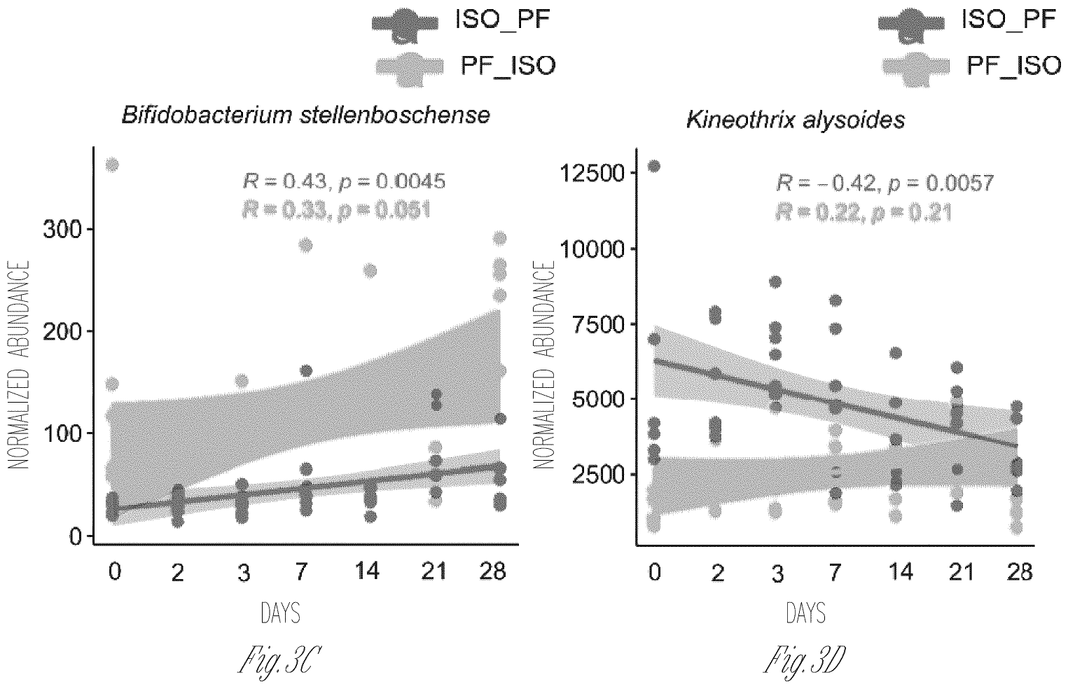

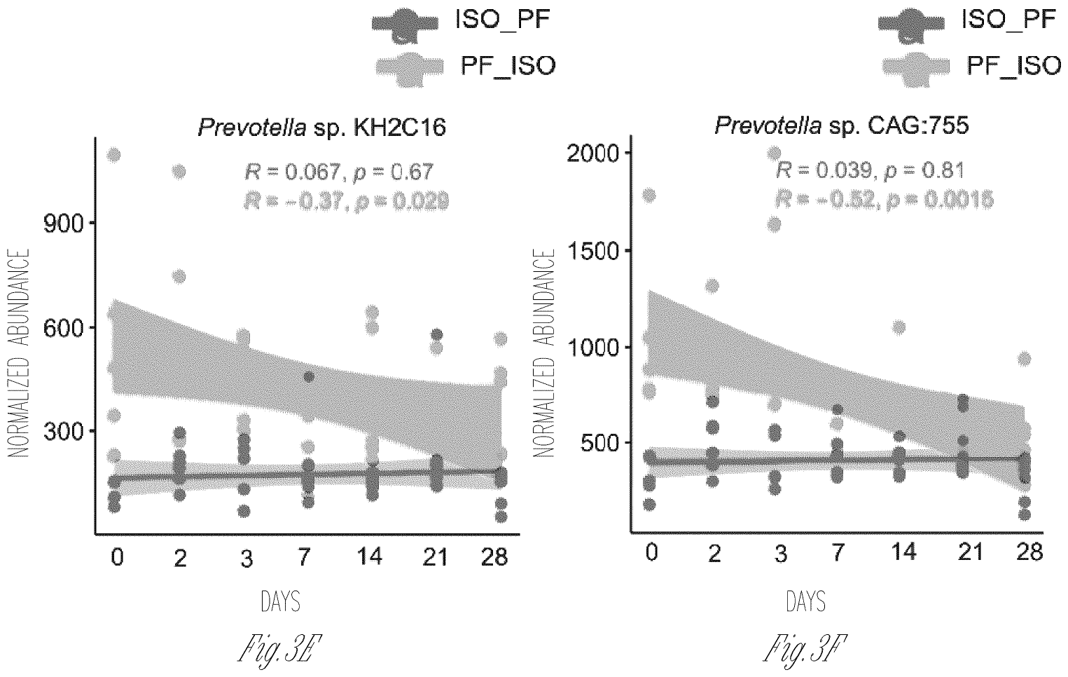

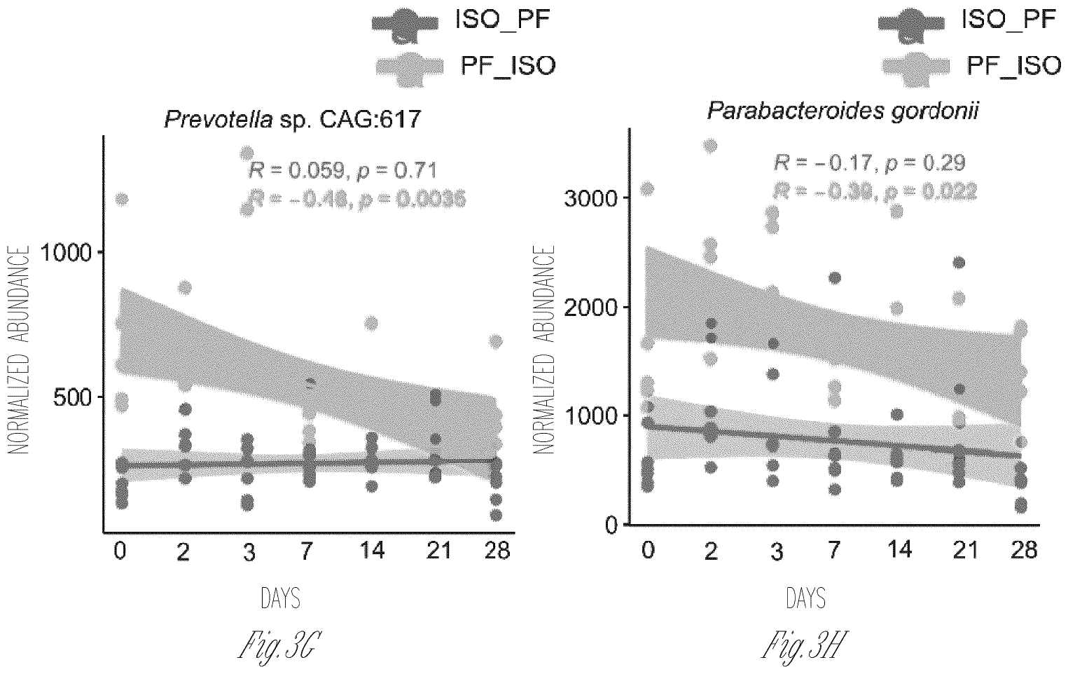

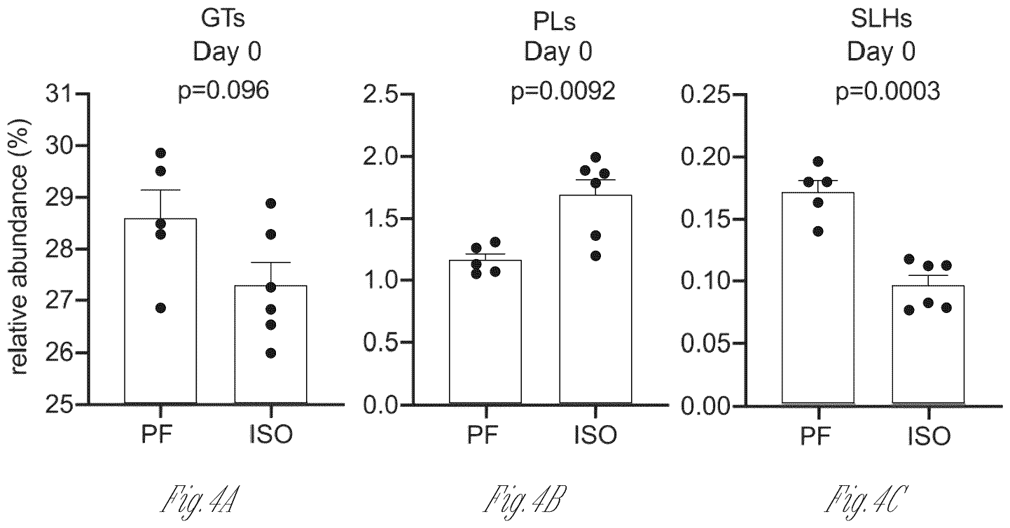

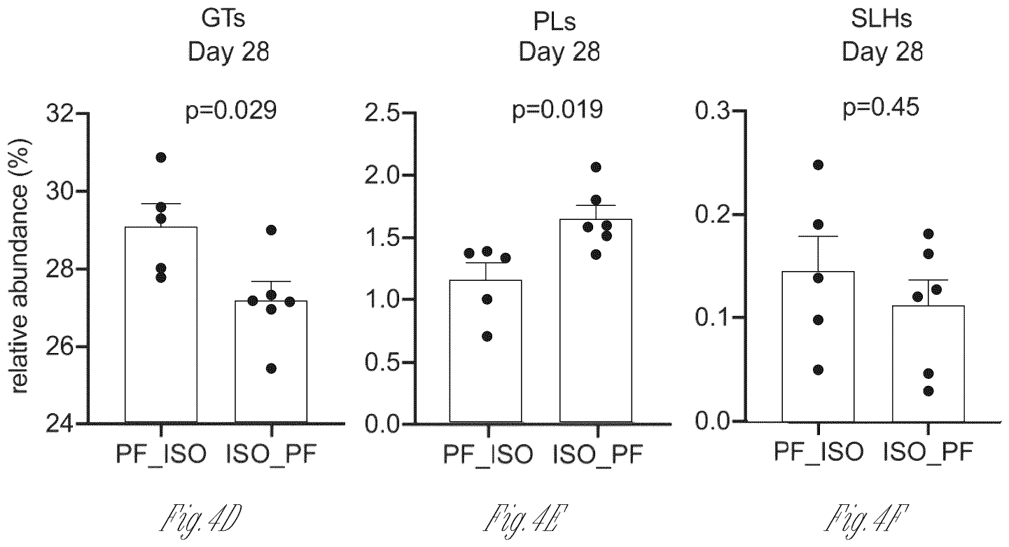

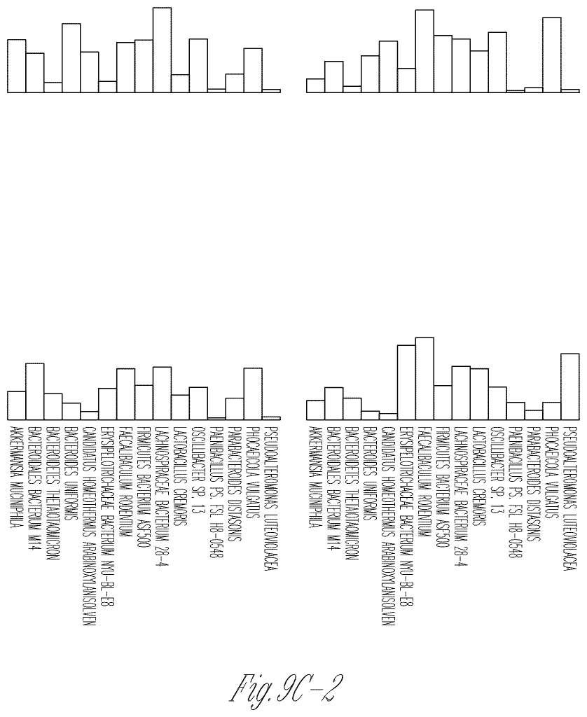

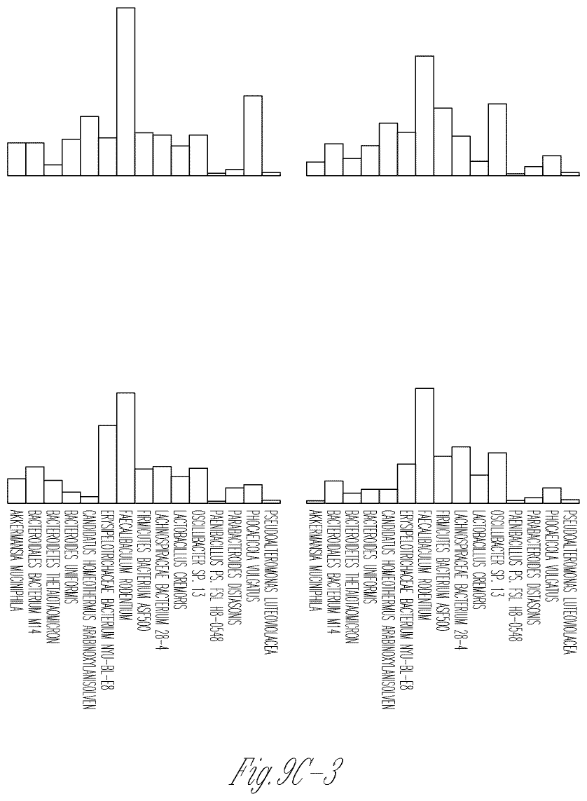

In addition to phytoestrogen metabolizing genes, the shift in gut microbiota composition of mice at baseline and mice after the diet switch on D28 was investigated. Taxonomically, Bacteroidetes and Firmicutes were the two major phyla of fecal microbial communities in mice kept on ISO or PF at D0 (FIG. 9a). There was no significant difference between the Bacteroidetes to Firmicutes ratio (B:F ratio) for the mice at D0 (p=0.79). These two phyla also dominated the gut after the diet change in both groups over 28 days. However, change to either diet brought a reduction in the Bacteroidetes population (FIG. 9a). This change was evident with a significant decrease in the B:F ratio over time (FIG. 9b). The reduction of B:F ratio was more pronounced when the diet was switched from PF to ISO. At the species level, Faecalibacterium rodentium, Phocaeicola vulgatus, and Erysipelotrichaceae bacterium NYU-BL-E8 were dominant in both PF and ISO dietary conditions at D0. The top dominant species with >5% abundance in the metagenomes on days 2, 3, 7, 14, 21, and 28 are shown in FIG. 9c. Upon differential analysis at D0, bacteria enriched in the PF diet belonged exclusively to either Bacteroidetes or Proteobacteria phylum (FIG. 2a). However, at least one member of bacteria belonging to Firmicutes, Actinobacteria, Bacteroidetes, and Verrucomicrobia were found in mice fed with the ISO diet (FIG. 2a). At the species level, Odoribacter splanchnicus. Bacteroides sps, Rhodaspirillum sps, and Rikenella microfusus were highly differentially abundant in the PF diet whereas Akkermansia muciniphila, Bifidobacterium adolescentis, Bacteroides thetaiotaomicron, Candidatus homeotherum arabinoxylanisolvens, Kineothrix alysoides, Firmicutes bacterium CAG:102 and B. longum were differentially abundant in ISO diet at D0 (FIG. 2a). After the diet was switched, on D28, the majority of differentially expressed bacteria remained unaffected (FIG. 2b). However, the significance in the abundance of B. adolescentis. B. thetaiotaomicron, K. alysoides, Firmicutes bacterium CAG:102, and B. longum were lost after the diet was changed from ISO to PF. Also, Marinifilum fragile, Desulfomicrobium escambiense, Prevotella sp. KH2C16, Rikenellaceae bacterium DTU002, Bacteroidales bacterium Bact_10, Prevotella sp. CAG:755, Bacteroidetes bacterium GWD2_33_33, Desulfurispirillum indicum, Prevotella sp. CAG:617 and Reichenbachiella agariperforans were lost after 28 days of diet change to ISO. Instead, B. pseudocatemulatum, B. stellenboschense, Parabacteroides gordonii, Roseburia sp. CAG:309, Desulfovibrio cuneatus and D. gigas were differentially abundant on D28 after the diet was changed to ISO from PF (FIG. 2b). Among these differentially abundant taxa, eight species were significantly altered over time with diet change (FIG. 3). Specifically, B. adolescentis, B. longum and K. alysoides were significantly reduced with a change in diet from ISO to PF while B. stellenboschense was significantly increased (FIG. 3a, 3b, 3c, 3d). The change in diet from PF to ISO did not alter the abundance of these bacteria over time except for a significant increase of B. stellenboschense. However, the change from PF to ISO saw a significant reduction in the abundance of Prevotella sp. KH2C16, Prevotella sp. CAG:755, Prevotella sp. CAG:617 and Parabacteroides gordonii (FIG. 3e, 3f, 3g, 3h). As Bifidobacteria are designated as useful “probiotic” species beneficial for the human gut (Sullivan & Nord, 2005; Gibson, 1999; Tsangalis et al., 2002), the loss of these species after the diet change to PF is indicative of deterioration in the overall gut microbiome homeostasis.

### Dietary Change Modifies Lipopolysaccharides Biosynthesis by Gut Microbiota

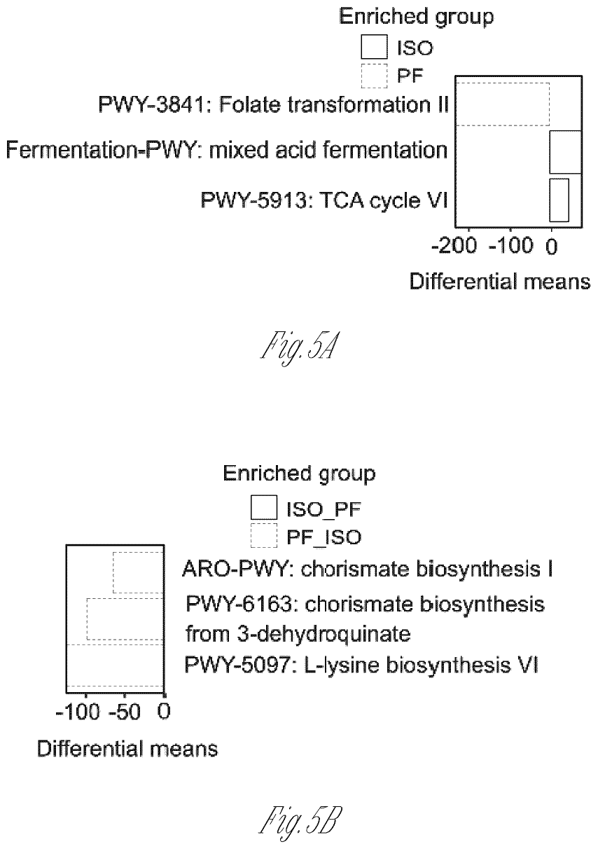

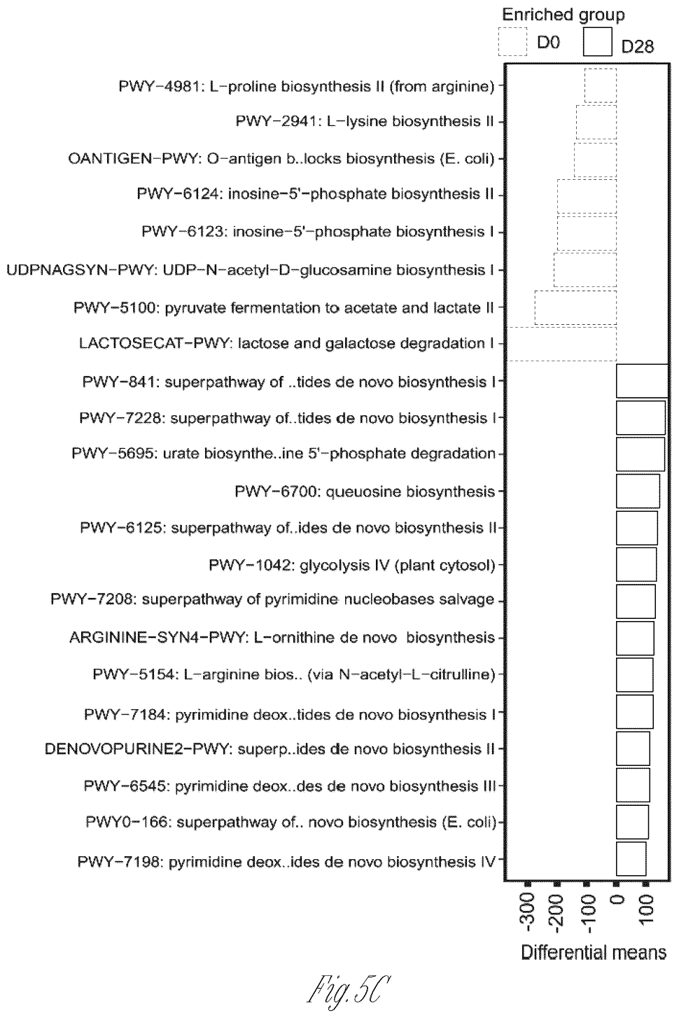

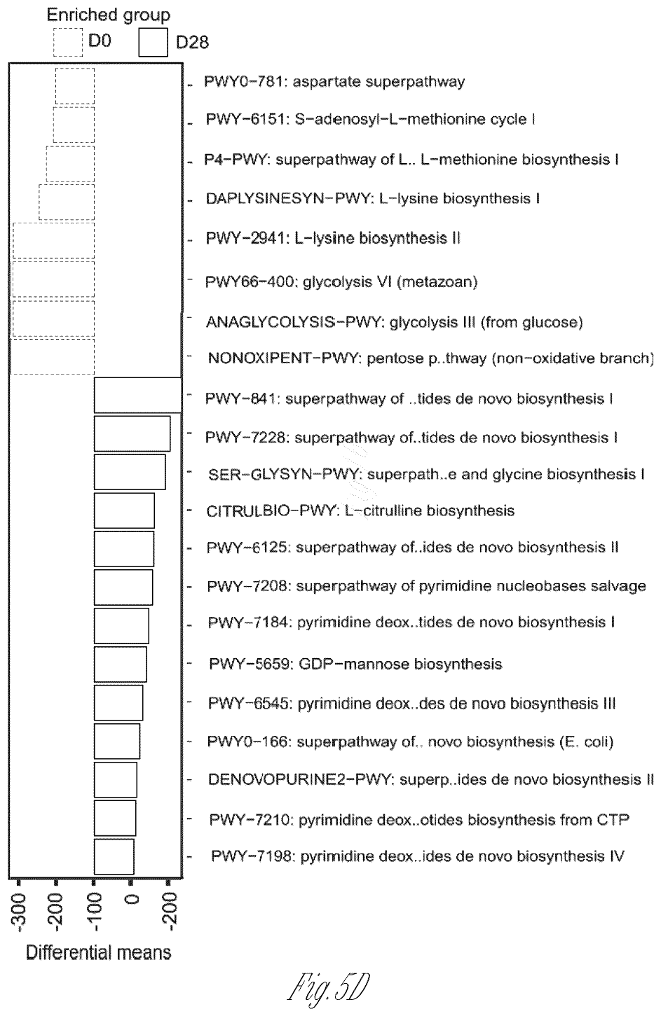

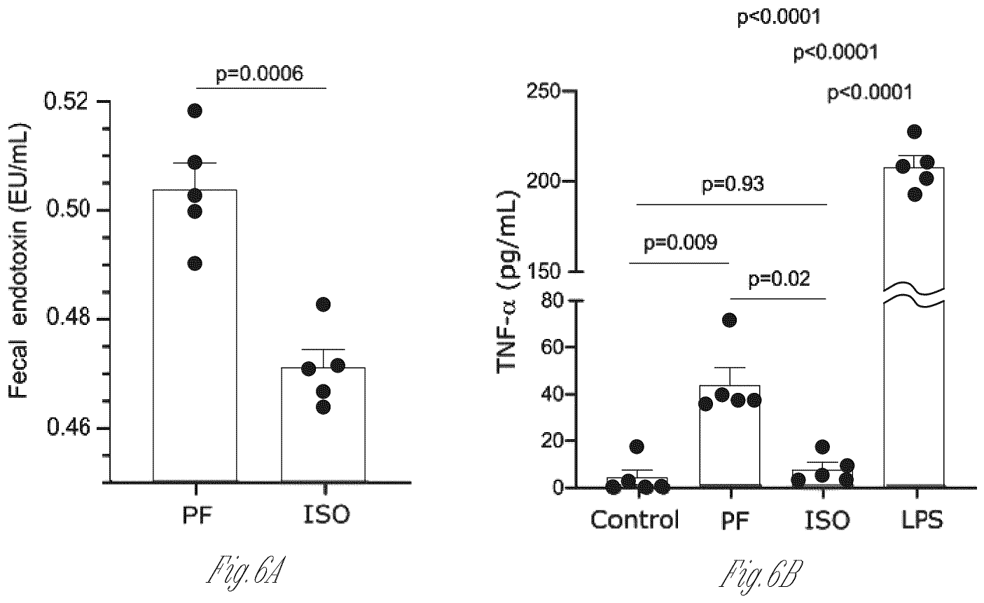

The presence of active enzymes present in the metagenomes of the microbiota before the diet change (D0) and on D28 after the diet change in both groups (FIG. 4) was determined. The relative abundances of auxiliary activities (AAs, p=0.68), carbohydrate-binding modules (CBMs, p=0.17), carbohydrate esterases (CEs, p=0.33), glycoside hydrolases (GHs, p=0.16) and glycosyltransferases (GTs) (p=0.096) (FIG. 4a) were similar between the mice fed with ISO or PF at D0. However, mice fed with ISO diet had significantly higher polysaccharide lyase (PLs) activity along with a reduction of S-layer homology domains (SLHs) activity (FIG. 4b, 4c). No significant changes in the relative abundances of AAs, CBMs, CEs, and GHs were observed after 28 days of diet change between the groups. Yet, there was a significant reduction in GTs activity when the diet was changed from ISO to PF (FIG. 4d). Within GTs, only GT2_glyco_tranf_2 activity was significantly higher in diet change from PF to ISO compared to diet change from ISO to PF (p=0.00045) by D28. GT2 family of enzymes are diverse and are involved in transferring the sugar from UDP-glucose, UDP-N-acetyl-galactosamine, GDP-mannose, or CDP-abequose, to a range of substrates including cellulose, dolichol phosphate, and teichoic acids (https://pfam.xfam.org/family/PF00535) which form the building blocks of lipopolysaccharides implying that isoflavone dietary conditions may contribute to formation of structurally different LPS compared to phyto-free diet. Similarly, the relative abundance of PLs activity was significant on D28 when the diet was changed to PF from ISO, but the change was also evident on D0 indicating that PLs were not affected overall by the change in diet (FIG. 4e). The significance of high SLHs activity in the PF diet at D0 was lost by D28 when the diet was changed to ISO (FIG. 4f) suggesting that the ISO diet reduces SLHs activity. SLHs are shown to mediate the binding of exocellular proteins to the cell surface in vivo and in vitro (Lemaire et al., 1998; Olabarria et al., 1996; Lemaire et al., 1995) and thus can affect cellular signaling. Taken together, differences in active carbohydrate enzymatic activity after dietary change suggest differences in LPS and exocellular protein composition and structure of microbiota.

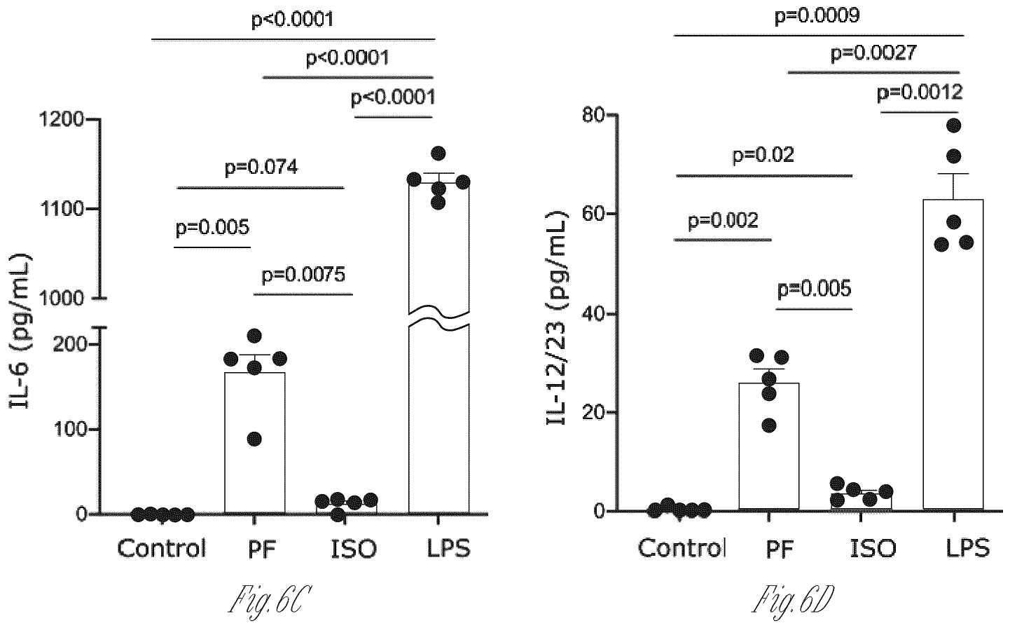

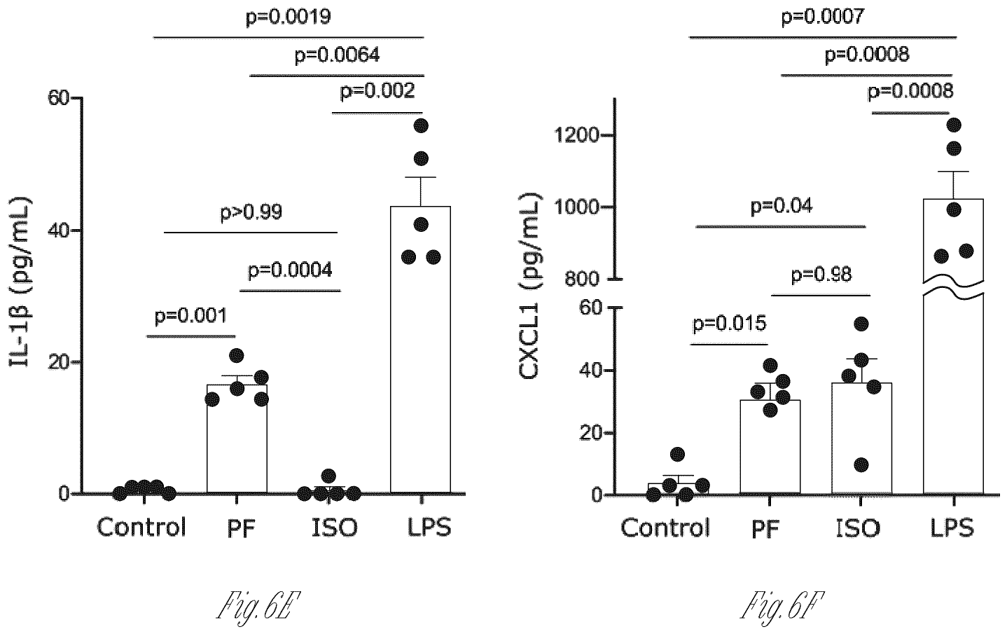

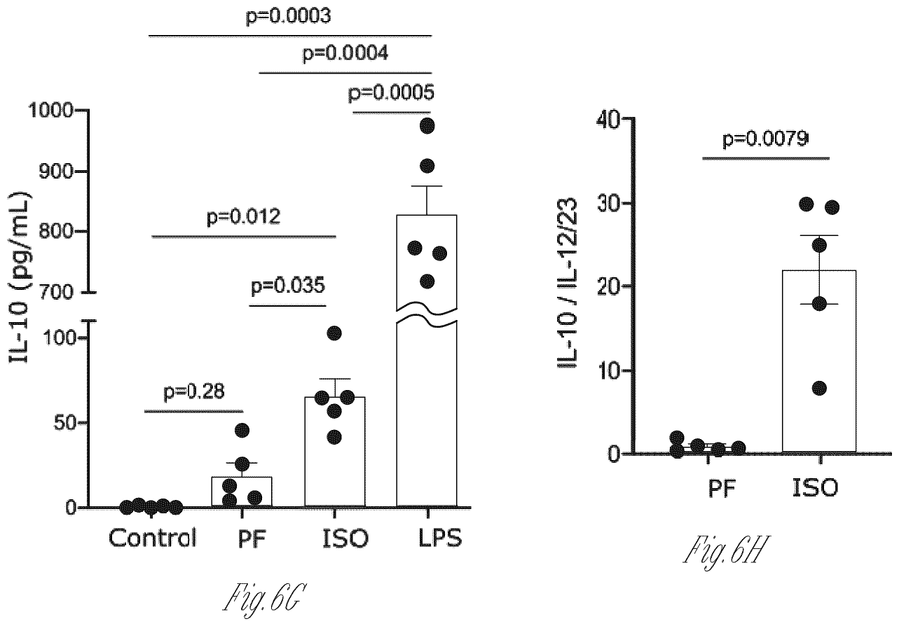

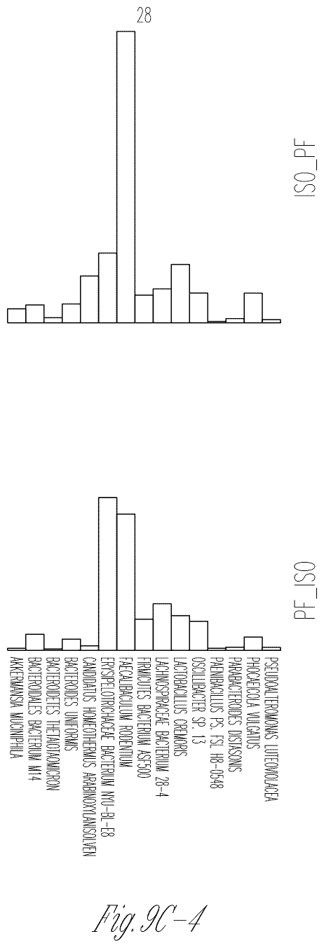

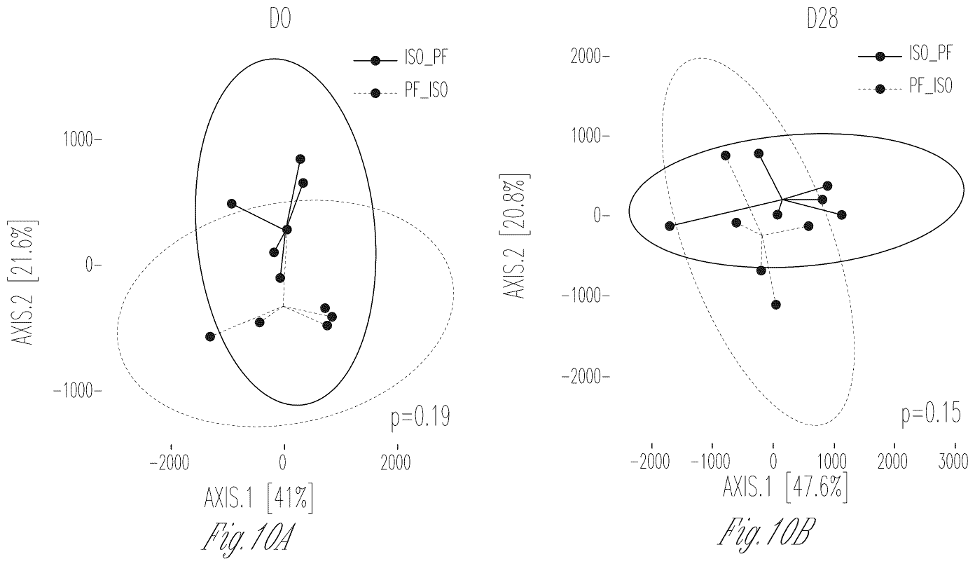

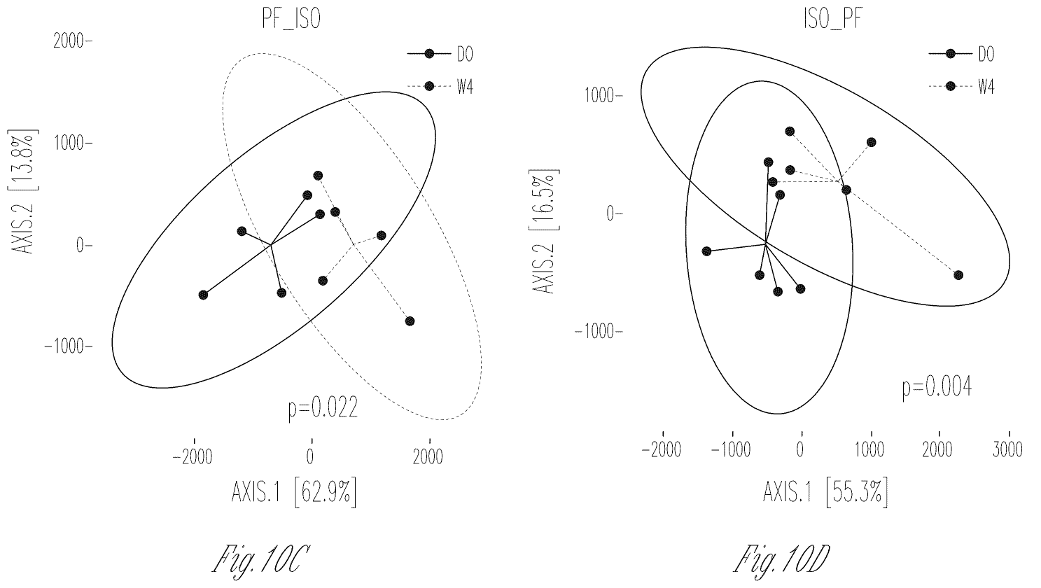

While no significant functional differences were found between the communities when comparing on D0 and D28 separately (FIG. 10a, 10b), there were significant differences between D0 and D28 over time for both diet changes (FIG. 10c, 10d). Functionally, on D0, the isoflavone diet had significant enrichment of mixed acid fermentation and TCA cycle VI pathways suggesting a higher abundance of short-chain fatty acid pathways (FIG. 5a). The folate transformation pathway was only significantly enriched on phyto-free diet. However, on D28, the diet change from PF to ISO had significant enrichment of chorismite biosynthesis pathways and L-lysine biosynthesis VI pathway (FIG. 5b). The lysine biosynthesis pathway VI is important as it produces an important metabolite meso-diaminopimelate which is a constituent of the bacterial cell wall peptidoglycan. The chorismite biosynthesis pathways produce chorismite which is an important intermediate leading to the synthesis of essential metabolites such as L-phenylalanine, L-tyrosine, L-tryptophan, Vit E and K, ubiquinone, and certain siderophores. Especially, these amino acids act as substrates for the production of secondary metabolites from alkaloids, flavonoids, lignin, coumarin, and other phenolic compounds implying that a diet switch to PF from ISO but not ISO from PF enhances secondary metabolite production from dietary flavonoids.

On D28, the diet change from ISO to PF led to enriched lactose and galactose degradation, pyruvate fermentation, and inosine-5′ phosphate biosynthesis pathways along with UDP-N-acetyl-D-glucosamine biosynthesis I and O-antigen building blocks biosynthesis pathways compared to D0 (FIG. 5c). UDP-N-acetyl-D-glucosamine is the precursor of cell wall peptidoglycan, lipopolysaccharide, and enterobacterial common antigen. Similarly, the O-antigen specific chains are a part of lipopolysaccharides that are known to evoke a specific immune response. Thus, it appears that the diet switch from ISO to PF enhances lipopolysaccharides biosynthesis in the gut to induce an immune response. However, no such LPS biosynthesis pathway enrichment was observed when the diet was changed to PF from ISO on D28 when compared to baseline D0. As the observed differences in the carbohydrate enzymatic activity were after dietary change which could produce differences in the LPS and exocellular protein structure produced by the microbiota, lipopolysaccharide synthesis is likely altered because of dietary ISO or PF conditions after the diet change. In fact, the differences in the gut microbiota has been previously described to affect immune signaling and immunogenicity in humans because of their LPS differences (Vatanen et al., 2016). Also, enrichment of methionine, aspartate, and lysine pathways along with glycolysis and non-oxidant pentose phosphate pathways on D28 compared to D0 was observed when the diet was switched from PF to ISO (FIG. 5d). It indicates that diet change to PF alters gut microbiota functions and may promote the synthesis of LPS-based antigenic molecules to evoke a different immune response.

### Isoflavone Diet Promotes Anti-Inflammatory Cytokines

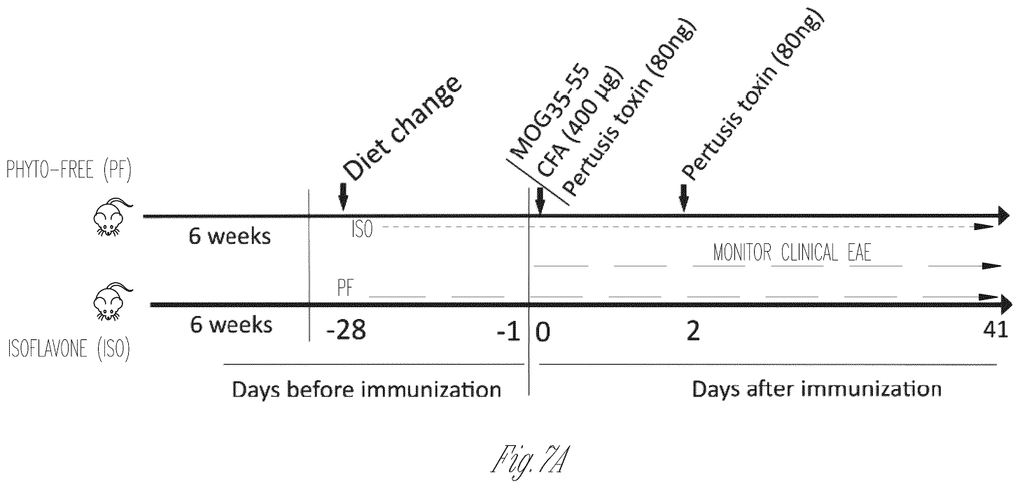

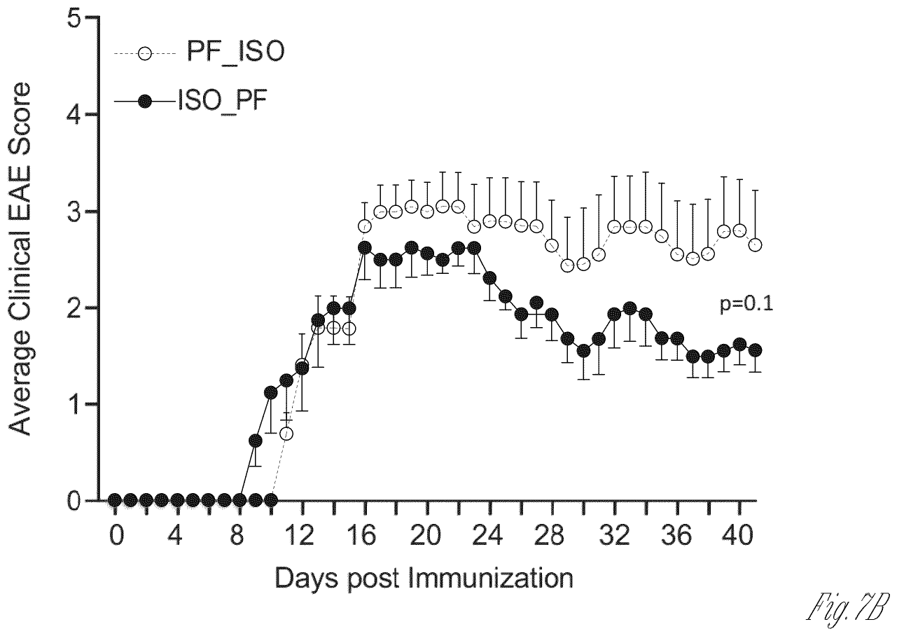

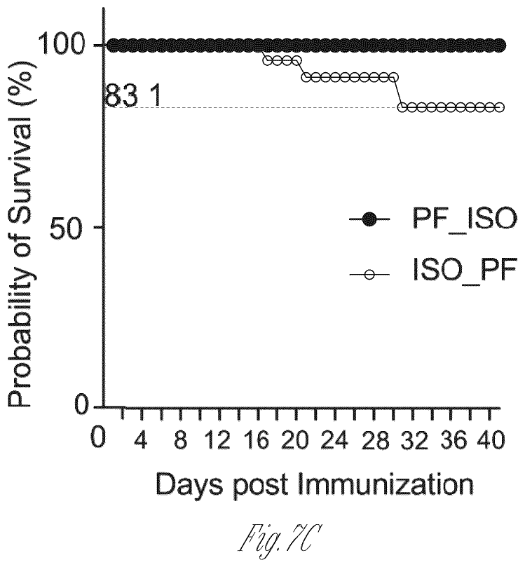

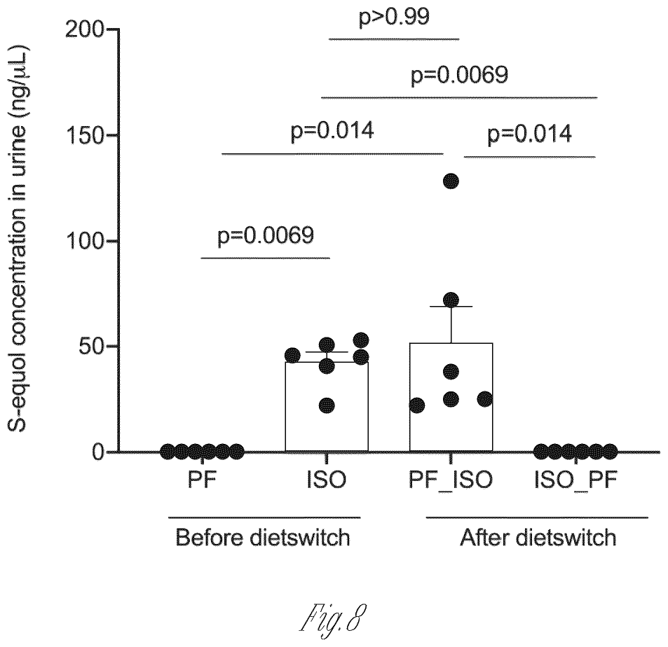

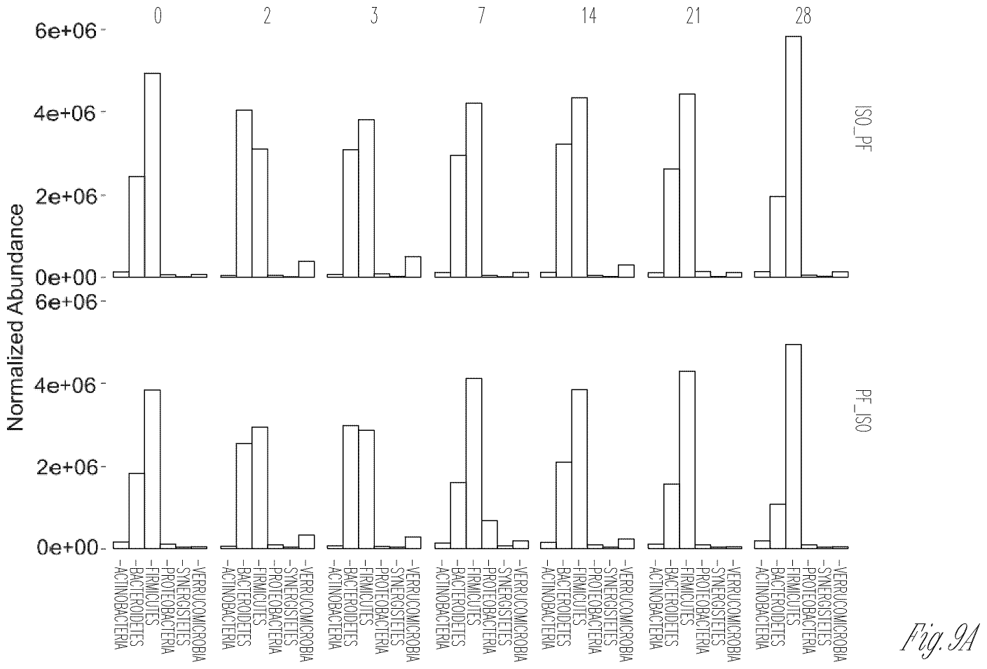

From the functional analysis of the gut microbiota, an alteration of LPS biosynthesis along with an increase in O-antigen biosynthesis (E. coli) and UDP-N-acetyl-D-glucosamine biosynthesis pathway after four weeks were observed when the diet was switched from ISO to PF. First, the fecal endotoxin levels in the fecal lysate obtained from mice from both groups were quantified. It was observed that the fecal endotoxin levels were significantly higher in mice kept on PF diet (FIG. 6a). To test the effect of the differences in the pro/anti-inflammatory effect of LPS synthesized after diet switch, bone marrow-derived macrophages were treated with LPS isolated from the fecal lysate of mice kept on either PF or ISO for four weeks, and quantified expression of the cytokines TNF-alpha, IL-6, IL-1beta, CXCL1, IL-10 and IL-12/23 in the supernatant. The LPS derived from the feces of mice kept on the PF diet produced significantly higher levels of TNF-alpha, IL-6, IL-12/23 and IL-beta compared to the LPS derived from feces of mice kept on the ISO diet (FIG. 6b, 6c, 6d, 6e). There was no difference in the CXCL1 levels between the PF and ISO diet fed groups (FIG. 6f). On the other hand, IL-10 production was significantly higher in the mice kept on ISO diet (FIG. 6g). Higher levels of TNF-alpha, IL-6, IL-1beta and IL-12/23 and lower levels of IL-10 indicate that the PF diet may contributes to a pro-inflammatory effect in the gut. Additionally, the ratio of IL-10 to IL-12/23 in BMDM induced with LPS of the feces of mice kept on either PF or ISO was significantly higher for mice kept on ISO diet (FIG. 6h) highlighting the potent anti-inflammatory effect of ISO diet altered LPS. These results suggest pro-inflammatory consequences for mice fed with PF diet and anti-inflammatory outcomes of the ISO diet in the mouse gut.

Diet Change from Phyto-Free to Isoflavone Reduces EAE Severity

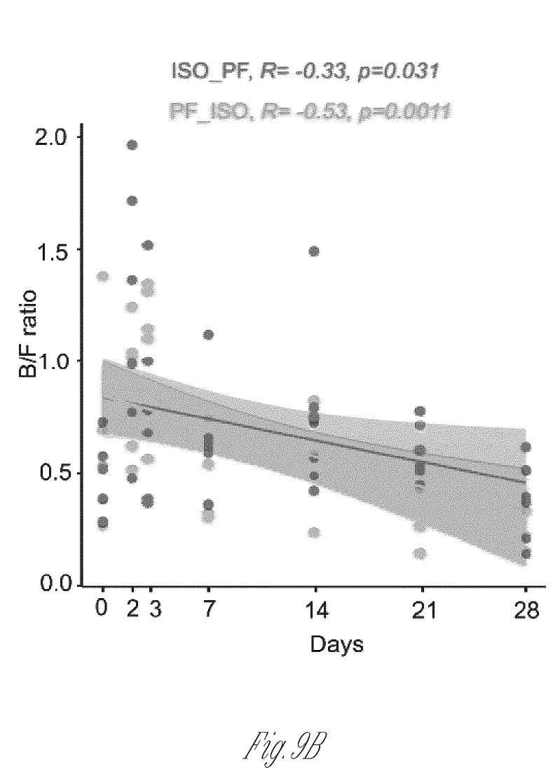

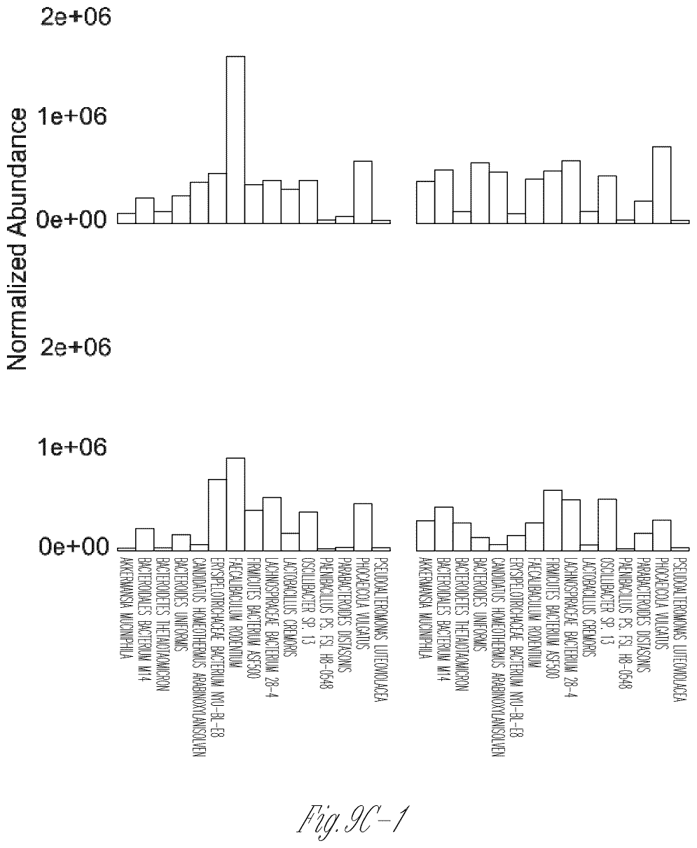

It was previously shown that the isoflavone diet significantly reduces EAE severity (Jensen et al., 2021). Here, it was observed that the ISO diet exerts an anti-inflammatory effect. Thus, it was investigated if the diet change from PF to ISO or ISO to PF can affect EAE. EAE was induced in mice after 4 weeks of diet change as shown in FIG. 7a. After EAE induction mice were monitored for the EAE scores for 41 days. The onset of disease for the mice originally on the PF diet but later switched to ISO showed earlier disease onset compared to mice on the ISO diet originally and later switched to the PF diet (FIG. 7b). However, mice switched to the ISO diet from the PF diet had consistently trended towards lower average daily clinical scores over 41 days after immunization compared to mice in which diet was switched to PF from the ISO diet indicating that diet switch to ISO helps to reduce EAE severity (FIG. 7c). Interestingly, the probability of survival was lower (83.1%) in mice whose diet was switched to PF from ISO compared to mice whose diet was switched to ISO from PF indicating that diet switch to ISO confers protection from both EAE severity and fatality (FIG. 7c). Thus, the data suggest that a diet rich in isoflavone may suppress EAE disease severity and overall disease mortality compared to mice switched to a phyto-free diet.

### Discussion

As PwMS have gut dysbiosis with depletion of beneficial bacteria, restoring their microbiota toward a healthy microbiota is being explored as a potential treatment option. Diet has been shown to have the strongest effect on the composition of gut bacteria (Rothschild et al., 2018; Zhang et al., 2010) and thus can provide an exciting option to correct gut dysbiosis in pwMS as well as other diseases. It was previously shown that PwMS lack bacteria with the ability to metabolize dietary phytoestrogen and most importantly, that an isoflavone diet can suppress disease in an animal model of MS (Jensen et al., 2021). The disease protection was dependent on the presence of isoflavone metabolizing bacteria and their metabolite. Thus, patients with inflammatory diseases may be treated with phytoestrogens including isoflavone. Therefore, the extent to which the dietary regimen change to isoflavone rich (ISO) or phyto-free (PF) affects the microbiota composition and its functions, and its effect on disease modulation, is determined. Here, it was demonstrated that changing the diet from phyto-free to isoflavone enhances evenness and phylogenetic diversity in the gut microbiota. The data reveal a shift in microbial communities and functional capabilities after a short-term diet switch which affected lipopolysaccharide biosynthesis. Furthermore, the impact of differences in the LPS produced after the diet switch was evident as we observed enhanced anti-inflammatory cytokine production when the diet was changed from PF to ISO compared to diet change from ISO to PF. The change in the microbial structure and functions ultimately affected the phenotypic outcome by reducing the production of pro-inflammatory cytokines and the severity of EAE after the diet was changed from PF to ISO. The data on the ability of an isoflavone-rich diet to regulate pro-inflammatory responses and protect from CNS inflammatory disease through modulation of LPS biosynthesis pathways points toward a previously unknown mechanism through which isoflavones can modulate host physiology. Taken together, these results provide proof that switching diet from PF to ISO can reduce pro-inflammatory responses and disease severity through the previously unknown effects of phytoestrogen metabolism on LPS biosynthesis.

Analyzing the gut microbiota of pwMS and healthy control samples, multiple studies have reported that several gut bacteria are differentially abundant in MS patients compared to healthy controls (Chen et al., 2016; Jangi et al., 2016). The abundance of gut bacteria that can metabolize isoflavones such as Adlercreutzia equolifaciens and Parabacteroides distasonis are reduced in MS patients indicating that isoflavone metabolism may be compromised in MS patients (Chen et al., 2016; Mangalam et al., 2021). Moreover, the isoflavone diet can ameliorate EAE in several mouse models (Jensen et al., 2021). Similarly, the current study showed that the change of diet from PF to ISO reduces pro-inflammatory responses, EAE severity, and mortality in mice compared to diet change from ISO to PF, confirming that isoflavones are protective against EAE. Moreover, diet change from PF to ISO altered gut microbiota and enhanced phylogenetic richness without affecting the microbial evenness. However, the diet change from ISO to PF significantly reduced the evenness of the microbiota and did not alter the phylogenetic diversity. These findings validate previous results where it was demonstrated that the isoflavone diet enhances species richness (Jensen et al., 2021). Indeed, healthy individuals harbor diverse gut bacteria. The increase in microbial diversity has been linked to gut homeostasis and overall better health (Shreiner et al., 2015). Similarly, the microbiome composition showed a longitudinal shift after the diet change for both conditions over time. The change in microbiota was evident as it resulted in the loss of isoflavone metabolizing genes as early as day 7 after the diet change from ISO to PF was made. These isoflavone metabolizing genes convert isoflavones to S-equol and were absent at D0 for the PF fed mice but were later found in some mice after day 28 of diet change to ISO. This finding suggests that microbial species harboring these genes are affected by the diet change and thus might influence potential S-equol production.

S-equol is produced as a result of the breakdown of dietary isoflavones such as daidzein and genistein by gut bacteria (Matthies et al., 2009). S-equol production requires a complete set of enzymes: daidzein reductase, dihydrodaidzein reductase, and tetrahydrodaidzein reductase to metabolize daidzein to S-equol (Kawada et al., 2016). Genistein was found to be catalyzed at an even higher rate by daidzein reductase compared to daidzein (Kawada et al., 2018). A previous study showed that both isoflavone metabolizing bacteria and their product is required for EAE suppression (Jensen et al., 2021). Even though rodents can efficiently produce S-equol from phytoestrogen metabolism (Brown & Setchell, 2001; Thigpen et al., 1999), well known bacterial “equol producers” were not found such as Adlercreutzia equolifaciens (Maruo et al., 2008), Eggerthella species (Kim et al., 2009; Yokoyama & Suzuki, 2008), Slackia isoflavoniconvertens (Schroder et al., 2013) and Lactococcus garvieae (Uchiyama et al., 2007) that were differentially enriched at baseline between the ISO and PF fed mice or at endpoint after the diet change was found. However, a significant loss of B. adolescentis and B. longum after the diet change to PF and gain of B. stellenboschense after the diet change to ISO. Bifidobacterium species were previously described to efficiently produce S-equol from isoflavones (Elghali et al., 2012) suggesting a loss of S-equol production after the diet change from ISO to PF. Importantly, the loss of S-equol levels after diet switch from ISO to PF validated that dietary change was critical for restoration of S-equol levels. Albeit the presence of homologs of isoflavone metabolizing enzymes in reference genomes of B. adolescentis, B. longum, and B. stellenboschense was not found. This indicated that genomes of these bacteria need further investigation for the presence of non-canonical genes responsible for metabolizing isoflavone. Also, on day 28 after diet was switched from PF to ISO, restoration of complete set of genes required for isoflavone degradation to S-equol as shown in Table 1 was not observed. However, S-equol levels were restored indicating that S-equol production is revived after diet change to ISO. Also, S-equol production rate is very low in human intestines and only 30-50% of humans can convert isoflavones to S-equol (Liu et al., 2010; Rowland et al., 2000). This implies that either there are undefined pathways for the production of S-equol or there are other possible methods by which dietary isoflavones are acted upon by gut microbes to benefit overall health.

Bifidobacteria are designated as useful “probiotic” species for the human gut and harbor beta-glucosidase enzyme activity required for initial hydrolysis of isoflavone glucosides (Sullivan & Nord, 2005; Gibson, 1999; Tsangalis et al., 2002), suggesting that the isoflavone diet likely selected a metabolic niche of beta-glucosidase activity. Also, Bifidobacterium species are better known for their diverse carbohydrate metabolism, SCFAs production, interaction with the host, secondary metabolite production, and probiotic effect that influence inflammation and homeostasis (Bottacini et al., 2014). In addition, B. adolescentis and B. longum species can produce exopolysaccharides which increase the production of anti-inflammatory cytokines such as IL-10 in the gut (Yu et al., 2019; Yan et al., 2019) suggesting that diet change might have contributed to alter polysaccharide assimilation by the gut microbiota. Indeed, it was observed a significant reduction in Bifidobacterium species along with changes in CHO-active enzymes: glycosyltransferase, polysaccharide lyase, and S-Layer-homology activity after 28 days of diet change. Also, the diet change to PF permitted specific enrichment of certain lipopolysaccharide biosynthesis pathways: UDP-N-acetyl-D-glucosamine biosynthesis I and O-antigen building blocks biosynthesis. Differences in microbiota have been previously described to affect immune signaling due to their LPS differences (Vatanen et al., 2016). These findings prompted us to investigate the properties of LPS present in the gut microbiota for both diets. Interestingly, treatment of bone marrow-derived macrophages with LPS derived from mice fed with the isoflavone diet showed significantly higher anti-inflammatory IL-10 and lower proinflammatory TNF-alpha, IL-6, IL-1beta and IL-12/23 cytokine production. In fact, feeding phytoestrogens has been shown to increase the anti-inflammatory effect in the CNS (Jensen et al., 2021) and LPS induced macrophages (Dia et al., 2008; Abron et al., 2018). Similarly, it was found that a significant loss of K. alysoides: a saccharolytic butyrate producer (Haas & Blanchard, 2017) which may promote a proinflammatory environment in the gut when the diet was changed to PF from ISO. Butyrate has been shown to increase the effectiveness and production of circulating Treg cells in mice which is important for peripheral tolerance to prevent autoimmune diseases (Smith et al., 2013). On other hand, Prevotella species were lost after the diet was switched to ISO. The loss of Prevotella species could be due to nutritional niche alteration or nutritional competition. Prevotella species occurrence is highly debated in terms of the role they play in health and diseases. The abundance of Prevotella species were found to decrease in MS patients (Chen et al., 2016) and importantly, P. histicola was shown to suppress EAE (Shahi et al., 2019). However, Prevotella species have been associated with several other inflammatory diseases and are proposed as a marker of the definite state of microbiome promoting inflammation rather than cause (Iljazovic et al., 2021; Lucke et al., 2006). Taken, together, these findings suggest an alternative mechanism by which the isoflavone diet supports the enrichment of beneficial bacteria and alters lipopolysaccharide assimilation to modulate the EAE severity in mice. Although diet switch from PF to ISO resulted in lower average clinical scores of EAE, it did not reach statistical significance. Thus, it is possible that a longer timeframe after the diet switch is required for significant disease suppression.

In conclusion, a change in diet to isoflavones induced a change in gut microbiota composition and LPS production pathways. Such changes were evident with ISO-induced LPS enhancing the expression of anti-inflammatory cytokines regardless of presence of the isoflavone metabolizing bacteria. This lays a justification for how isoflavones can be beneficial through their ability to induce S-equol production and alteration of LPS biosynthesis.

The shotgun metagenomic sequences are deposited in NCBI under BioProject PRJNA834824.

### Example III

Mice on a Healthy (Isoflavone Diet) Diet have a Higher Ratio of Bifidobacterium Adolescentis to Akkermansia muciniphila than Mice on a Phytoestrogen-Free Diet

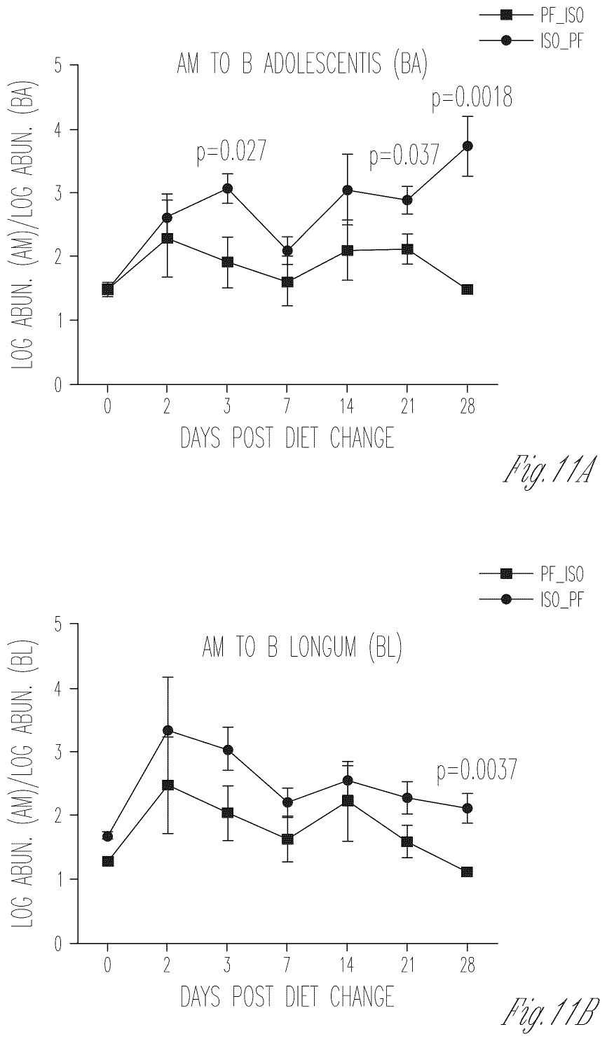

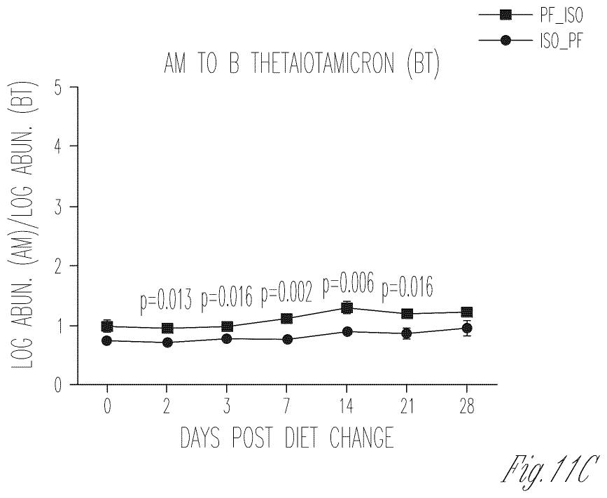

A change in diet from isoflavone (ISO) to phytoestrogen-free diet (PF) significantly reduced the abundance of Bifidobacterium adolescentis (BA), Bifidobacterium longum (BL) and Bacteroides thetaiotamicron (W) without affecting levels of A muciniphila (Ghimire et al 2022). The data pointed towards a scenario where the presence or absence of beneficial bacteria might define the symbiont (beneficial bacteria) vs. pathobiont (disease promoting) properties of a commensal such as A muciniphila (AM). Based on the data, the ratio of a symbiont to a pathobiont rather than the presence or absence of a commensal might may determine the pro vs. anti-inflammatory nature of A muciniphila. Therefore, the ratio of AM abundance with the abundance of other symbiotics that were lost in mice where the diet was switched from ISO To PF or vice-versa was determined. This was indeed true as Bifidobacterium spp. especially, B. adolescentis showed enrichment over time when the diet was switched from PF to ISO and a reduced abundance when switched from ISO to PF overtime. Mice switched from an ISO to PF diet (ISO-PF) showed a higher AM/BA ratio (AM/BA, AM/BL, and AM/BT ratio) (FIG. 11). Thus, the data suggests that the absence of isoflavone (phytoestrogen) results in a higher ratio of AM to beneficial bacteria, especially Bifidobacterium adolescentis.

These findings combined with other studies that mice on a phytoestrogen-free (PF) diet show proinflammatory properties, suggest that the absence of isoflavone results in the loss of beneficial bacteria and might promote the transition of AM from a commensal to a pathobiont.

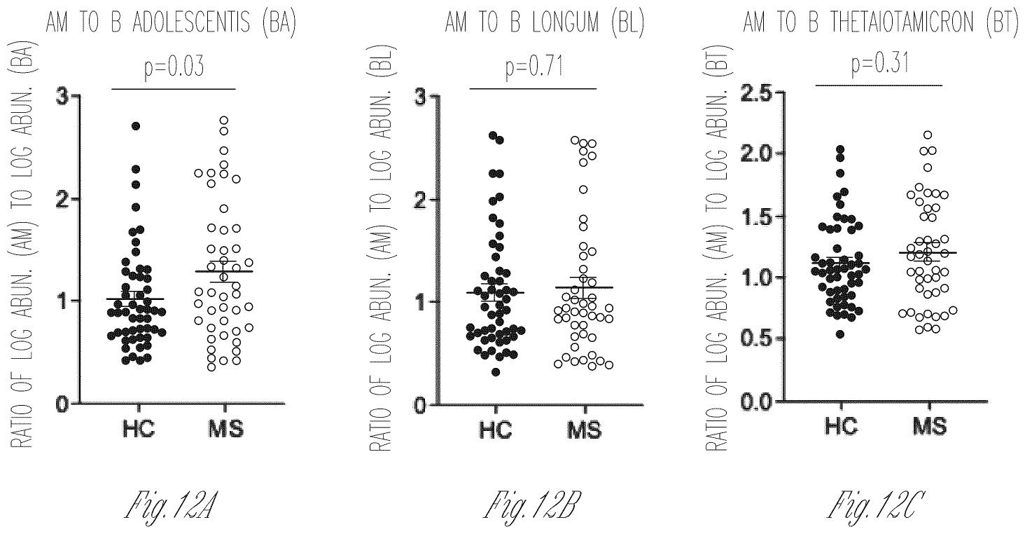

Data on the differential bacterial ratio from the preclinical animal model in patients with Multiple Sclerosis. To determine the clinical significance of gut bacteria that show higher abundance in mice on ISO-D but are depleted when switched to PF diet, the ratio of AM to these specific bacteria in the fecal samples from people with MS (PwMS) (n=45) and healthy control (HC) (n=51) were analyzed using shotgun metagenomic sequencing (FIG. 12). Interestingly, among all bacterial species analyzed, only Bifidobacterium adolescentis (BA) showed a significantly higher AM/Bifidobacterium ratio (FIG. 12). This data, combined with the finding that BA showed a difference between ISO and PF groups at baseline (FIG. 11) and AM/BA ratio being significantly different at more time points than AM to BT or BL ratio (data not shown), indicate that abundance of BA might be the critical gut bacteria responsible for mediating the beneficial effects of phytoestrogen.

### REFERENCES

Abron et al., PLoS One, 13:e0199631 (2018).
Abuqwider et al., Microorganisms, 9:______(2021).
Adamberg et al., Benef. Microbes, 9:21 (2018).
Allaire et al., Trends Immunol., 39:677 (2018).
Bailey et al., Nitric Oxide, 100:17 (2020).
Beghini et al., Elife, 10:______(2021).
Bibbo et al., Eur. Rev. Med. Pharmacol. Sci., 20:4742 (2016).
Bottacini et al., Microbial Cell Factories, 13:S4 (2014).
Bowey et al., Food Chem. Toxicol., 41:631 (2003).
Branca & Lorenzetti, Forum Nutr., 57:100 (2005).
Breugelmans et al., Lancet Gastroenterol. Hepatol., 7:455 (2022).
Brown & Setchell, Lab. Invest., 81:735 (2001).
Brushett et al., Front Pediatr., 8:408 (2020).
Cady et al., Front Neurol., 11:150 (2020).
Camara-Lemarroy et al., World J. Gastroenterol., 24:4217 (2018).
Cantoni et al., EBioMedicine, 76:103798 (2022).
Cekanaviciute et al., Proc. Natl. Acad. Sci. USA, 114:10713 (2017).
Chen et al., Sci. Rep., 6:28484 (2016).
Chien et al., Food Microbiol., 23:772 (2006).
Chu et al., Mediators Inflamm., 2018:8168717 (2018).
Colpitts et al., Gut Microbes, 8:561 (2017).
Confavreux et al., N. Engl. J. Med., 339:285 (1998).
Cosorich et al., Sci. Adv., 3: e1700492 (2017).
Davanzo et al., Mult. Scler. Relat. Disord., 72:104605 (2023).
den Besten et al., J. Lipid Res., 54:2325 (2013).
Derrien et al., Appl. Environ. Microbiol., 74:1646 (2008).
Dia et al., J. Agric. Food Chem., 56:11707 (2008).
Drula et al., Nucleic Acids Res., 50:D571 (2022).
Ebers et al., N. Engl. J. Med., 3:1638 (1986).
Elghali et al., J. Funct. Foods, 4:736 (2012).
Forgie et al., Front. Immunol., 10:1802 (2019).
Gensollen et al., 352:539 (2016).
Ghimire et al., Gut. Microbes, 14:2127446 (2022).
Ghimire et al., mSystems, 5:______(2020).
Gibson, J. Nutr., 129:1438S (1999).
Giordano et al., Am. J. Pharmacogenomics, 2:37 (2002).
Goodin, D. S., PLoS One, 4:e4565 (2009).
Haas & Blanchard, Int. J. Syst. Evol. Microbiol., 67:402 (2017).
Hagi & Belzer, Appl. Microbiol. Biotechnol., 105:4833 (2021).
Hansen et al., Mult. Scler., 11:504 (2005).
Heng et al., Arch. Microbiol., 201:1009 (2019).
Hertog et al., Lancet, 342:1007 (1993).
Hidalgo-Cantabrana et al., Microbiol. Spectr., 5:______(2017).
Hoftberger & Lassmann, Handb. Clin. Neurol., 145:263 (2017).
Human Microbiome Project, Nature, 486:207 (2012).
Iljazovic et al., Int. J. Med. Microbiol., 311:151472 (2021).
iMSMS, Cell, 185:3467 e16 (2022).
Irish et al., Degener. Neurol. Neuromuscul. Dis., 7:1 (2017).
Jangi et al., Nat. Commun., 7:12015 (2016).
Jangi et al., Nat. Commun., 7:12015 (2016).
Jensen et al., Sci. Adv., 7:______(2021).
Ji et al., Front. Immunol., 10:1881 (2019).
Kawada et al., Biosci. Microbiota Food Health, 35:113 (2016).
Kawada et al., J. Biosci. Bioeng., 126:301 (2018).
Kim et al., Appl. Environ. Microbiol., 75:3062 (2009).
Kim et. al., BMC Microbiol., 16:9 (2016).
Koch et al., J. Neurol. Sci., 324:10 (2013).
Kuusisto et al., Eur. J. Neurol., 15:1106 (2008).
Laffont et al., Biomed. J., 38:194 (2015).
Lang, Clin. Immunol., 113:224(2004).
Larrosa et al., Mol. Nutr. Food Res., 53:1044 (2009).
Lemaire et al., J. Bacteriol., 177:2451 (1995).
Lemaire et al., Microbiology (Reading), 144:211 (1998).
Li et al., Bioinformatics, 31:1674 (2015).
Liu et al., J. Epidemiol., 20:377 (2010).
Liu et al., Cell Host Microbe, 2:779 (2019).
Lucke et al., J. Med. Microbiol., 55:617 (2006).
Mangalam et al., Ann. NY Acad, Sci., 1103:112 (2007).
Mangalam et al., Indian J. Rheumatol., 16:57 (2021).
Marcais et al., PLoS Comput. Biol., 14:e1005944 (2018).
Maruo et al., Int. J. Syst. Evol. Microbiol., (2008) 58:1221-7.
Maslowski & Mackay, Nat. Immunol., 12:5 (2011).
Matthies et al., Appl. Environ. Microbiol., 75:1740 (2009).
Mayo et al., Nutrients, 11:______ (2019).
McGill et al., J. Neuroimmunol., 342:577209 (2020).
Mende et al., Nucleic Acids Res., 45:D529 (2017).
Menzel et al., Nat. Commun., 7:11257 (2016).
Milani et al., Appl. Environ. Microbiol., 82:980 (2016).
Miyake et al., PLoS One, 10:e0137429 (2015).
Morito et al., Biol. Pharm. Bull., 24:351 (2001).
Natividad & Verdu, Pharmacol. Res., 69:42 (2013).
Offner & Polanczyk, Ann. NY Acad. Sci. 1089:343 (2006).
Olabarria et al., J. Bacteriol., 178:4765 (1996).
Palmas et al., Sci. Rep., 11:5532 (2021).
Peddada, Science, 357:754 (2017).
Peterson et al., ImmunoHorizons, ______:______ 2023.
Qin et al., Nat. Genet., 54:134 (2022).
R Core Team, R: A Language and Environment for Statistical Computing. Vienna, Austria: R Foundation for Statistical Computing (2021).
Raju et al., J. Chromatogr. B Analyt. Technol. Biomed. Life Sci., 1129:121776 (2019).
Rimbach et al., Food Chem. Toxicol., 46:1308 (2008).
Rinninella et al., Nutrients, 11:______(2019).
Rothschild et al., Nature, 555:210 (2018).
Rothschild et al., Nature, 555:210 (2018).
Rowland et al., Nutr. Cancer, 36:27 (2000).
Schroder et al., Appl. Environ. Microbiol., 79:3494 (2013).
Seemann, Bioinformatics, 30:2068 (2014).
Segura Munoz et al., ISME J., 16:1594 (2022).
Segura Munoz et al., Mol. Nutr. Food Res., 64:e2000162 (2020).
Shah et al., EBioMedicine, 2021. 71: p. 103557.
Shahi et al., Front Immunol., 10:462 (2019).
Shahi et al., Front Immunol., 13:966417 (2022).
Shi et al., J. Agric. Food Chem., 6:6333 (2020).
Shreiner et al., Curr. Opin. Gastroenterol., 31:69 (2015).
Shrode et al., Metabolomics, 18:84 (2022).
Smith et al., Science, 341:569 (2013).
Smits et al., Science, 3_7:802 (2017).
Sonnenburg & Backhed, Nature, 535:56 (2016).
Stoiloudis et al., Nutrients, 14:______(2022).
Sullivan & Nord, J. Intern. Med., 257:78 (2005).
Swain Ewald & Ewald, Yale J. Biol. Med., 91:445 (2018).
Thigpen et al., Lab. Anim. Sci., 49:530 (1999).
Thirion et al., Genome Med., 15:1 (2023).
Tiwari-Woodruff & Voskuhl, J. Neurol. Sci., 286:81 (2009).
Tiwari-Woodruff et al., Proc. Natl. Acad. Sci. USA, 104:14813 (2007).
Tremlett et al., Eur. J. Neurol., 23:1308 (2016).
Tsangalis et al., J. Food Sci., 67:3104 (2002).
Turroni et al., Cell Mol. Life Sci., 75:103 (2018a).
Turroni et al., Trends Microbiol., 26:339 (2018b).
Tyler et al., PLoS One, 8:e66772 (2013).
Uchiyama et al., J. Intest. Microb., 21:217 (2007).
Uritskiy et al., Microbiome, 6:158 (2018).
Valle Gottlieb et al., Crit. Rev. Food Sci. Nutr., 0.8:1557 (2018).
Vatanen et al., Cell 165:842 (2016).
Wahls, Phys. Med. Rehabil. Clin. N. Am., 33:605 (2022).
Wahts et al., Mult. Scler. J. Exp. Transl. Clin., 7:20552173211035399 (2021).
Walton et al., Mult. Scler., 26:1816 (2020).
Waubant et al., Ann. Clin. Transl. Neurol., 6:1905 (2019).
Willer et al., Proc. Natl. Acad. Sci. USA, 100:12877 (2003).
Yadav et al., PLoS One, 17:e0264556 (2022).
Yadav et al., Proc. Natl. Acad. Sci. USA, 114:E9318 (2017).
Yan et al., Food Funct., 10:1595 (2019).
Yan et al., Gut Microbes, 13:1984104 (2021).
Yang, microbiomeMarker: microbiome biomarker analysis toolkit (2020).
Yokoyama & Suzuki, Biosci. Biotechnol. Biochem., 22:2660 (2008).
Yu et al., Nutrients, 11:______(2019).
Zhang et al., ISME J., 4:232 (2010).
Zhang et al., Nucleic Acids Res., 46:W95 (2018).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to prevent, inhibit or treat an inflammatory disease in a mammal, comprising: administering to a mammal in need thereof an effective amount of one or more isolated gut microbes and one or more phytoestrogens.

2. The method of claim 1 wherein the mammal is a human.

3. The method of claim 1 wherein the mammal has an autoimmune disease.

4. (canceled)

5. The method of claim 1 wherein at least one of the gut microbes comprises Adlercreutzia, or Parabacteroides.

6-8. (canceled)

9. The method of claim 1 wherein the one or more phytoestrogens comprise a coumestan, a prenylflavonoid, a lignan, an isoflavonoid, isoflavanol, genistein, quercetin, glycitin, daidzein, O-DMA (desmethylangolensin) or S-equol.

10-16. (canceled)

17. The method of claim 1 any one of claims 1 to 16 wherein the one or more gut microbes or the one or more phytoestrogens are orally administered.

18-19. (canceled)

20. A method to decrease disease severity in a mammal, comprising: administering to a mammal in need thereof an effective amount of one or more isolated gut microbes and one or more phytoestrogens.

21. The method of claim 20 wherein disease is an inflammatory disease.

22. The method of claim 20 wherein the mammal is obese.

23. The method of claim 20 wherein the mammal has cancer.

24. The method of claim 20 wherein the mammal has an autoimmune disease.

25. The method of claim 20 wherein the mammal is a human.

26. The method of claim 20 wherein at least one of the gut microbes comprises Parabacteroides or Adlercreutzia.

27. The method of claim 20 wherein the one or more phytoestrogens comprise isoflavanol, genestein, quercetin, glycitin, O-DMA, daidzein or S-equol.

28. The method of claim 20 wherein the one or more gut microbes and the one or more phytoestrogens are administered concurrently.

29. The method of claim 20 wherein the one or more gut microbes and/or wherein the one or more phytoestrogens are orally administered.

30-39. (canceled)

40. A package comprising a first composition comprising an amount of one or more isolated gut microbes and a second composition comprising an amount of one or more phytoestrogens, wherein one of the microbes comprises Parabacteroides or Adlercreutzia.

41. The package of claim 40 wherein the first composition, the second composition, or both, are formulated for oral or sustained delivery.

42-46. (canceled)
